(19) United States
(12) Patent Application Publication
(10) Pub. No.: US 2010/0035259 A1
(43) Pub. Date: Feb. 11, 2010

(54) BIOMARKERS AND METHODS FOR DETERMINING SENSITIVITY TO VASCULAR ENDOTHELIAL GROWTH FACTOR RECEPTOR-2 MODULATORS

(75) Inventors: Shujian Wu, Langhorne, PA (US); Mark David Ayers, Pennington, NJ (US); Xia Han, Pennington, NJ (US); Suganthy Yoganathan, Clinton, NJ (US); Joseph Fargnoli, Pipersville, PA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 12/450,122

(22) PCT Filed: Mar. 12, 2008
(86) PCT No.: PCT/US2008/003258

§ 371 Date: Sep. 11, 2009

Related U.S. Application Data
(60) Provisional application No. 60/906,433, filed on Mar. 12, 2007.

(51) Int. Cl. C12Q 1/68

(52) U.S. Cl. 435/6

(57) ABSTRACT

VEGFR-2 biomarkers useful in a method for identifying and monitoring a mammal that will respond therapeutically to a method of treating cancer comprising administering an VEGFR-2 modulator, wherein the method comprises (a) exposing the mammal to the VEGFR-2 modulator and (b) measuring in the mammal the level of the at least one biomarker, wherein a difference in the level of the at least one biomarker measured in (b) compared to the level of the biomarker in a mammal that has not been exposed to the VEGFR-2 modulator indicates that the mammal will respond therapeutically to the method of treating cancer and (c) wherein the level of the biomarker in a mammal after exposure to a VEGFR-2 modulator indicates that the mammal has responded therapeutically to the method of treating cancer

### SEQUENCE LISTING

A compact disc labeled “Copy 1” contains the Sequence Listing as 10918 PCT.ST25.txt. The Sequence Listing is 1262 KB in size and was recorded Mar. 12, 2008. The compact disk is 1 of 2 compact disks. A duplicate copy of the compact disc is labeled “Copy 2” and is 2 of 2 compact discs.

The compact disc and duplicate copy are identical and are hereby incorporated by reference into the present application.

### FIELD OF THE INVENTION

The present invention relates generally to the field of pharmacogenomics, and more specifically, to methods and procedures used to monitor response or determine sensitivity in patients to allow the identification of individualized genetic profiles which will aid in treating diseases and disorders.

### BACKGROUND OF THE INVENTION

Cancer is a disease with extensive histoclinical heterogeneity. Although conventional histological and clinical features have been correlated to prognosis, the same apparent prognostic type of tumors varies widely in its responsiveness to therapy and consequent survival of the patient.

New prognostic and predictive markers, which would facilitate an individualization of therapy for each patient, are needed to accurately predict patient response to treatments, such as small molecule or biological molecule drugs, in the clinic. The problem may be solved by the identification of new parameters that could better predict the patient's sensitivity to treatment. The classification of patient samples is a crucial aspect of cancer diagnosis and treatment. The association of a patient's response to a treatment with molecular and genetic markers can open up new opportunities for treatment development in non-responding patients, or distinguish a treatment's indication among other treatment choices because of higher confidence in the efficacy. Further, the pre-selection of patients who are likely to respond well to a medicine, drug, or combination therapy may reduce the number of patients needed in a clinical study or accelerate the time needed to complete a clinical development program (M. Cockett et al., Current Opinion in Biotechnology, 11:602-609 (2000)).

The ability to determine which patients are responding to anti-angiogenesis therapies (such as VEGFR-2 modulators) or predict drug sensitivity in patients is particularly challenging because drug responses reflect not only properties intrinsic to the target cells, but also a host's metabolic properties. Efforts to use genetic information to predict or monitor drug response have primarily focused on individual genes that have broad effects, such as the multidrug resistance genes mdr1 and mrp1 (P. Sonneveld, J. Intern. Med., 247:521-534 (2000)).

The development of microarray technologies for large scale characterization of gene mRNA expression pattern has made it possible to systematically search for molecular markers and to categorize cancers into distinct subgroups not evident by traditional histopathological methods (J. Khan et al., Cancer Res., 58:5009-5013 (1998); A. A. Alizadeh et al., Nature, 403:503-511 (2000); M. Bittner et al., Nature, 406:536-540 (2000); J. Khan et al., Nature Medicine, 7(6):673-679 (2001); and T. R. Golub et al., Science, 286:531-537 (1999); U. Alon et al., P. N. A. S. USA, 96:6745-6750 (1999)). Such technologies and molecular tools have made it possible to monitor the expression level of a large number of transcripts within a cell population at any given time (see, e.g., Schena et al., Science, 270:467-470 (1995); Lockhart et al., Nature Biotechnology, 14:1675-1680 (1996); Blanchard et al., Nature Biotechnology, 14:1649 (1996); U.S. Pat. No. 5,569,588 to Ashby et al.).

Recent studies demonstrate that gene expression information generated by microarray analysis of human tumors can predict clinical outcome (L. J. van't Veer et al., Nature, 415:530-536 (2002); M. Shipp et al., Nature Medicine, 8(1):68-74 (2002); G. Glinsky et al., The Journal of Clin. Invest, 113(6):913-923 (2004)). These findings bring hope that cancer treatment will be vastly improved by better predicting and monitoring the response of individual tumors to therapy.

PCT Application No. PCT/US2006/034201 provides biomarkers useful for identifying a mammal that will respond therapeutically to a method of treating cancer comprising administering an VEGFR-2 modulator.

Needed are new and alternative methods and procedures to determine drug sensitivity or monitor response in patients to allow the development of individualized diagnostics which are necessary to treat diseases and disorders based on patient response at a molecular level.

### SUMMARY OF THE INVENTION

The invention provides methods and procedures for determining patient sensitivity or monitor response at the molecular level to one or more vascular endothelial growth factor receptor 2 (VEGFR-2) modulators. The invention also provides methods of determining or predicting whether an individual requiring therapy for a disease state such as cancer will or will not respond to treatment, prior to administration of the treatment, wherein the treatment comprises administration of one or more VEGFR-2 modulators. The one or more VEGFR-2 modulators are compounds that can be selected from, for example, one or more VEGFR-2 specific ligands, one or more small molecule VEGFR-2 inhibitors, or one or more VEGFR-2 binding monoclonal antibodies.

In one aspect, the invention provides a method for identifying a mammal that will respond therapeutically to a method of treating cancer comprising administering an VEGFR-2 modulator, wherein the method comprises: (a) measuring in the mammal the level of at least one biomarker selected from the biomarkers of Tables 2-4; (b) exposing a biological sample from the mammal to the VEGFR-2 modulator; (c) following the exposing in step (b), measuring in said biological sample the level of the at least one biomarker, wherein a difference in the level of the at least one biomarker measured in step (c) compared to the level of the at least one biomarker measured in step (a) indicates that the mammal will respond therapeutically to said method of treating cancer.

A difference in the level of the biomarker that is sufficient to indicate whether the mammal will or will not respond therapeutically to the method of treating cancer can be readily determined by one of skill in the art using known techniques. The increase or decrease in the level of the biomarker can be correlated to determine whether the difference is sufficient to identify a mammal that will respond therapeutically. The difference in the level of the biomarker that is sufficient can, in one aspect, be predetermined prior to determining whether the mammal will respond therapeutically to the treatment. In one aspect, the difference in the level of the biomarker is a difference in the mRNA level (measured, for example, by RT-PCR or a microarray), such as at least a two-fold difference, at least a three-fold difference, or at least a four-fold difference in the level of expression. In another aspect, the difference in the level of the biomarker is determined by IHC. In another aspect, the difference in the level of the biomarker refers to a p-value of <0.05 in Anova analysis. In yet another aspect, the difference is determined in an ELISA assay.

As used herein, respond therapeutically refers to the alleviation or abrogation of the cancer. This means that the life expectancy of an individual affected with the cancer will be increased or that one or more of the symptoms of the cancer will be reduced or ameliorated. The term encompasses a reduction in cancerous cell growth or tumor volume. Whether a mammal responds therapeutically can be measured by many methods well known in the art, such as PET imaging.

The mammal can be, for example, a human, rat, mouse, dog rabbit, pig sheep, cow, horse, cat, primate, or monkey.

The method of the invention can be, for example, an in vitro method wherein the step of measuring in the mammal the level of at least one biomarker comprises taking a biological sample from the mammal and then measuring the level of the biomarker(s) in the biological sample. The biological sample can comprise, for example, at least one of serum, whole fresh blood, peripheral blood mononuclear cells, frozen whole blood, fresh plasma, frozen plasma, urine, saliva, skin, hair follicle, bone marrow, or tumor tissue.

The level of the at least one biomarker can be, for example, the level of protein and/or mRNA transcript of the biomarker(s).

In another aspect, the invention provides a method for identifying a mammal that will respond therapeutically to a method of treating cancer comprising administering an VEGFR-2 modulator, wherein the method comprises: (a) exposing a biological sample from the mammal to the VEGFR-2 modulator; (b) following the exposing of step (a), measuring in said biological sample the level of at least one biomarker selected from the biomarkers of Tables 2-4, wherein a difference in the level of the at least one biomarker measured in step (b), compared to the level of the biomarker in a mammal that has not been exposed to said VEGFR-2 modulator, indicates that the mammal will respond therapeutically to said method of treating cancer.

In yet another aspect, the invention provides a method for testing or predicting whether a mammal will respond therapeutically to a method of treating cancer comprising administering an VEGFR-2 modulator, wherein the method comprises: (a) measuring in the mammal the level of at least one biomarker selected from the biomarkers of Tables 2-4; (b) exposing the mammal to the VEGFR-2 modulator; (c) following the exposing of step (b), measuring in the mammal the level of the at least one biomarker, wherein a difference in the level of the at least one biomarker measured in step (c) compared to the level of the at least one biomarker measured in step (a) indicates that the mammal will respond therapeutically to said method of treating cancer.

In another aspect, the invention provides a method for determining whether a compound inhibits VEGFR-2 activity in a mammal, comprising: (a) exposing the mammal to the compound; and (b) following the exposing of step (a), measuring in the mammal the level of at least one biomarker selected from the biomarkers of Tables 2-4, wherein a difference in the level of said biomarker measured in step (b), compared to the level of the biomarker in a mammal that has not been exposed to said compound, indicates that the compound inhibits VEGFR-2 activity in the mammal.

In yet another aspect, the invention provides a method for determining whether a mammal has been exposed to a compound that inhibits VEGFR-2 activity, comprising (a) exposing the mammal to the compound; and (b) following the exposing of step (a), measuring in the mammal the level of at least one biomarker selected from the biomarkers of Tables 2-4, wherein a difference in the level of said biomarker measured in step (b), compared to the level of the biomarker in a mammal that has not been exposed to said compound, indicates that the mammal has been exposed to a compound that inhibits VEGFR-2 activity.

In another aspect, the invention provides a method for determining whether a mammal is responding to a compound that inhibits VEGFR-2 activity, comprising (a) exposing the mammal to the compound; and (b) following the exposing of step (a), measuring in the mammal the level of at least one biomarker selected from the biomarkers of Tables 2-4, wherein a difference in the level of the at least one biomarker measured in step (b), compared to the level of the at least one biomarker in a mammal that has not been exposed to said compound, indicates that the mammal is responding to the compound that inhibits VEGFR-2 activity.

As used herein, “responding” encompasses responding by way of a biological and cellular response, as well as a clinical response (such as improved symptoms, a therapeutic effect, or an adverse event), in a mammal

The invention also provides an isolated biomarker selected from the biomarkers of Tables 2-4. The biomarkers of the invention comprise sequences selected from the nucleotide and amino acid sequences provided in Tables 2-4 and the Sequence Listing, as well as fragments and variants thereof.

The invention also provides a biomarker set comprising two or more biomarkers selected from the biomarkers of Tables 2-4.

The invention also provides kits for determining or predicting whether a patient would be susceptible or resistant to a treatment that comprises one or more VEGFR-2 modulators. The patient may have a cancer or tumor such as, for example, a colon cancer or tumor.

In one aspect, the kit comprises a suitable container that comprises one or more specialized microarrays of the invention, one or more VEGFR-2 modulators for use in testing cells from patient tissue specimens or patient samples, and instructions for use. The kit may further comprise reagents or materials for monitoring the expression of a biomarker set at the level of mRNA or protein.

In another aspect, the invention provides a kit comprising two or more biomarkers selected from the biomarkers of Tables 2-4.

In yet another aspect, the invention provides a kit comprising at least one of an antibody and a nucleic acid for detecting the presence of at least one of the biomarkers selected from the biomarkers of Tables 2-4. In one aspect, the kit further comprises instructions for determining whether or not a mammal will respond therapeutically to a method of treating cancer comprising administering a compound that inhibits VEGFR-2 activity. In another aspect, the instructions comprise the steps of (a) measuring in the mammal the level of at least one biomarker selected from the biomarkers of Tables 2-4, (b) exposing the mammal to the compound, (c) following the exposing of step (b), measuring in the mammal the level of the at least one biomarker, wherein a difference in the level of the at least one biomarker measured in step (c) compared to the level of the at least one biomarker measured in step (a) indicates that the mammal will respond therapeutically to said method of treating cancer.

The invention also provides screening assays for determining if a patient will be susceptible or resistant to treatment with one or more VEGFR-2 modulators.

The invention also provides a method of monitoring the treatment of a patient having a disease, wherein said disease is treated by a method comprising administering one or more VEGFR-2 modulators.

The invention also provides individualized genetic profiles which are necessary to treat diseases and disorders based on patient response at a molecular level.

The invention also provides specialized microarrays, e.g., oligonucleotide microarrays or cDNA microarrays, comprising one or more biomarkers having expression profiles that correlate with either sensitivity or resistance to one or more VEGFR-2 modulators.

The invention also provides antibodies, including polyclonal or monoclonal, directed against one or more biomarkers of the invention.

The invention will be better understood upon a reading of the detailed description of the invention when considered in connection with the accompanying figures.

### BRIEF DESCRIPTION OF THE FIGURES

FIG. 1 shows the expression values of ESM1 and MEST in stromal tissue upon either compound 1 or bevacizumab treatment at 17 Days.

FIG. 2 shows the expression values of S100A8 and S100A9 in stromal tissue upon either compound 1 or bevacizumab treatment at 17 Days.

FIG. 3 shows the expression value of EMP1 in tumor tissue upon either compound 1 or bevacizumab treatment at 17 Days.

FIG. 4 shows the expression values of VEGF, VEGFB, COL18A1, CEACAM1, and AKAP7 in tumor tissue upon either compound 1 or bevacizumab treatment at 17 Days.

### DETAILED DESCRIPTION OF THE INVENTION

Multiple preclinical studies have demonstrated the important role VEGF plays in driving the angiogenic process through its cognate receptors, the VEGFR family of transmembrane protein tyrosine kinases. The VEGFR-2 signaling pathway, in particular, has been experimentally supported to be a major driver of tumor angiogenesis. The VEGFR-2 signaling pathway is a clinically validated pathway in cancer therapy based on the approval of AVASTIN (bevacizumab), which indirectly inhibits this signaling pathway by preventing VEGF ligand binding. Compound 1, as defined below, has demonstrated potent inhibition of VEGFR-2 as well as inhibition of FGFR-1 and FGFR-2, another receptor family of protein tyrosine kinases underlying the angiogenic pathway and regulated by FGF growth factors. It is believed that by inhibiting multiple important angiogenesis pathways (VEGF and FGF), compound 1 has shown to have a increase affect on the ability to inhibit tumor growth when compared to bevacizumab in a GEO colon cancer model.

This biomarker study has lead to the identification of a set of biomarkers that reflect anti-angiogenesis and anti-tumor activities at the molecular level for which compound 1 has a greater affect relative to bevacizumab. The degree to which these biomarkers are affected is strongly associated with level of efficacy observed in this in vivo tumor model. These biomarkers could have important clinical implications in determining the optimal anti-angiogenesis therapy for cancer patients.

Identification of biomarkers that provide rapid and accessible readouts of efficacy, drug exposure, or clinical response is increasingly important in the clinical development of drug candidates. Embodiments of the invention include measuring changes in the levels of secreted proteins, or plasma biomarkers, which represent one category of biomarker. In one aspect, plasma samples, which represent a readily accessible source of material, serve as surrogate tissue for biomarker analysis.

The invention provides biomarkers that respond to the modulation of a specific signal transduction pathway and also correlate with VEGFR-2 modulator sensitivity or resistance. These biomarkers can be employed for predicting and monitoring response to one or more VEGFR-2 modulators. In one aspect, the biomarkers of the invention are those provided in Tables 2-4 and the Sequence Listing, including both polynucleotide and polypeptide sequences. In another aspect, the biomarkers of the invention are nucleotide sequences that, due to the degeneracy of the genetic code, encodes for a polypeptide sequence provided in the sequence listing.

The biomarkers serve as useful molecular tools for predicting and monitoring response to VEGFR-2 modulators that affect VEGFR-2 activity or the VEGFR-2 signal transduction pathway.

### VEGFR-2 Modulators:

As used herein, the term “VEGFR-2 modulator” is intended to mean a compound or drug that is a biological molecule or a small molecule that directly or indirectly modulates VEGFR-2 activity or the VEGFR-2 signal transduction pathway. Thus, compounds or drugs as used herein is intended to include both small molecules and biological molecules. Direct or indirect modulation includes activation or inhibition of VEGFR-2 activity or the VEGFR-2 signal transduction pathway. In one aspect, inhibition refers to inhibition of the binding of VEGFR-2 to an VEGFR-2 ligand such as, for example, VEGF. In another aspect, inhibition refers to inhibition of the kinase activity of VEGFR-2.

VEGFR-2 modulators include, for example, VEGFR-2 specific ligands, small molecule VEGFR-2 inhibitors, and VEGFR-2 monoclonal antibodies. In one aspect, the VEGFR-2 modulator inhibits VEGFR-2 activity and/or inhibits the VEGFR-2 signal transduction pathway. In another aspect, the VEGFR-2 modulator is an VEGFR-2 monoclonal antibody that inhibits VEGFR-2 activity and/or inhibits the VEGFR-2 signal transduction pathway.

VEGFR-2 modulators include biological molecules or small molecules.

Biological molecules include all lipids and polymers of monosaccharides, amino acids, and nucleotides having a molecular weight greater than 450. Thus, biological molecules include, for example, oligosaccharides and polysaccharides; oligopeptides, polypeptides, peptides, and proteins; and oligonucleotides and polynucleotides. Oligonucleotides and polynucleotides include, for example, DNA and RNA.

Biological molecules further include derivatives of any of the molecules described above. For example, derivatives of biological molecules include lipid and glycosylation derivatives of oligopeptides, polypeptides, peptides, and proteins.

Derivatives of biological molecules further include lipid derivatives of oligosaccharides and polysaccharides, e.g., lipopolysaccharides. Most typically, biological molecules are antibodies, or functional equivalents of antibodies. Functional equivalents of antibodies have binding characteristics comparable to those of antibodies, and inhibit the growth of cells that express VEGFR-2. Such functional equivalents include, for example, chimerized, humanized, and single chain antibodies as well as fragments thereof.

Functional equivalents of antibodies also include polypeptides with amino acid sequences substantially the same as the amino acid sequence of the variable or hypervariable regions of the antibodies. An amino acid sequence that is substantially the same as another sequence, but that differs from the other sequence by means of one or more substitutions, additions, and/or deletions, is considered to be an equivalent sequence. Preferably, less than 50%, more preferably less than 25%, and still more preferably less than 10%, of the number of amino acid residues in a sequence are substituted for, added to, or deleted from the protein.

The functional equivalent of an antibody is preferably a chimerized or humanized antibody. A chimerized antibody comprises the variable region of a non-human antibody and the constant region of a human antibody. A humanized antibody comprises the hypervariable region (CDRs) of a non-human antibody. The variable region other than the hypervariable region, e.g., the framework variable region, and the constant region of a humanized antibody are those of a human antibody.

Suitable variable and hypervariable regions of non-human antibodies may be derived from antibodies produced by any non-human mammal in which monoclonal antibodies are made. Suitable examples of mammals other than humans include, for example, rabbits, rats, mice, horses, goats, or primates.

Functional equivalents further include fragments of antibodies that have binding characteristics that are the same as, or are comparable to, those of the whole antibody. Suitable fragments of the antibody include any fragment that comprises a sufficient portion of the hypervariable (i.e., complementarity determining) region to bind specifically, and with sufficient affinity, to VEGFR-2 tyrosine kinase to inhibit growth of cells that express such receptors.

Such fragments may, for example, contain one or both Fab fragments or the F(ab′)2 fragment. Preferably, the antibody fragments contain all six complementarity determining regions of the whole antibody, although functional fragments containing fewer than all of such regions, such as three, four, or five CDRs, are also included.

In one aspect, the fragments are single chain antibodies, or Fv fragments. Single chain antibodies are polypeptides that comprise at least the variable region of the heavy chain of the antibody linked to the variable region of the light chain, with or without an interconnecting linker. Thus, Fv fragment comprises the entire antibody combining site. These chains may be produced in bacteria or in eukaryotic cells.

The antibodies and functional equivalents may be members of any class of immunoglobulins, such as IgG, IgM, IgA, IgD, or IgE, and the subclasses thereof.

In one aspect, the antibodies are members of the IgG1 subclass. The functional equivalents may also be equivalents of combinations of any of the above classes and subclasses.

In one aspect, the VEGFR-2 antibody is CDP-791 (UCB). In another aspect, the VEGFR-2 antibody is IMC-1121b (ImClone Systems). In yet another aspect, the VEGFR-2 modulator is AVE-005 (VEGF trap, Regeneron Pharmaceuticals).

In addition to the biological molecules discussed above, the VEGFR-2 modulators useful in the invention may also be small molecules. Any molecule that is not a biological molecule is considered herein to be a small molecule. Some examples of small molecules include organic compounds, organometallic compounds, salts of organic and organometallic compounds, saccharides, amino acids, and nucleotides. Small molecules further include molecules that would otherwise be considered biological molecules, except their molecular weight is not greater than 450. Thus, small molecules may be lipids, oligosaccharides, oligopeptides, and oligonucleotides and their derivatives, having a molecular weight of 450 or less.

It is emphasized that small molecules can have any molecular weight. They are merely called small molecules because they typically have molecular weights less than 450. Small molecules include compounds that are found in nature as well as synthetic compounds. In one embodiment, the VEGFR-2 modulator is a small molecule that inhibits the growth of tumor cells that express VEGFR-2. In another embodiment, the VEGFR-2 modulator is a small molecule that inhibits the growth of refractory tumor cells that express VEGFR-2.

Numerous small molecules have been described as being useful to inhibit VEGFR-2.

In one aspect, the VEGFR-2 modulator is [(1R), 2S]-2-Aminopropionic acid 2-[4-(4-fluoro-2-methyl-1H-indol-5-yloxy)-5-methylpyrrolo[2,1-f][1,2,4]triazin-6-yloxy]-1-methylethyl ester having the structure:

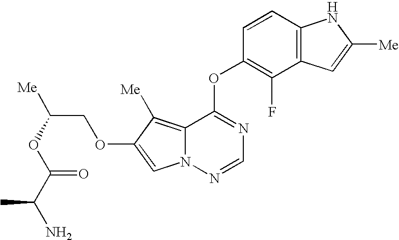

In another aspect, the VEGFR-2 modulator is selected from the compounds described in U.S. Pat. No. 6,869,952, hereby incorporated by reference. In yet another aspect, the VEGFR-2 modulator is selected from the compounds described in PCT Publication No. WO00/71129 or WO2004/009601, hereby incorporated by reference.

In another aspect, the VEGFR-2 modulator is selected from CHIR-258 (Chiron), AZD-2171 (AstraZeneca), GW786034 (GlaxoSmithKline), AMG 706 (Amgen), BIBF 1120 (Boehringer Ingelheim), AE788 (Novartis), ZD6474 (AstraZeneca), BAY 43-9006 (Sorafenib, Bayer), and SU11248 (Sutent, Pfizer).

### Biomarkers and Biomarker Sets:

The invention includes individual biomarkers and biomarker sets having both diagnostic and prognostic value in disease areas in which signaling through VEGFR-2 or the VEGFR-2 pathway is of importance, e.g., in cancers or tumors, in immunological disorders, conditions or dysfunctions, or in disease states in which cell signaling and/or cellular proliferation controls are abnormal or aberrant. The biomarker sets comprise a plurality of biomarkers such as, for example, a plurality of the biomarkers provided in Tables 2-4 that highly correlate with resistance or sensitivity to one or more VEGFR-2 modulators.

The biomarkers and biomarker sets of the invention enable one to predict or reasonably foretell the likely effect of one or more VEGFR-2 modulators in different biological systems or for cellular responses. The biomarkers and biomarker sets can be used in in vitro assays of VEGFR-2 modulator response by test cells to predict in vivo outcome. In accordance with the invention, the various biomarkers and biomarker sets described herein, or the combination of these biomarker sets with other biomarkers or markers, can be used, for example, to predict and monitor how patients with cancer might respond to therapeutic intervention with one or more VEGFR-2 modulators.

A biomarker and biomarker set of cellular gene expression patterns correlating with sensitivity or resistance of cells following exposure of the cells to one or more VEGFR-2 modulators provides a useful tool for screening one or more tumor samples before treatment with the VEGFR-2 modulator. The screening allows a prediction of cells of a tumor sample exposed to one or more VEGFR-2 modulators, based on the expression results of the biomarker and biomarker set, as to whether or not the tumor, and hence a patient harboring the tumor, will or will not respond to treatment with the VEGFR-2 modulator.

The biomarker or biomarker set can also be used as described herein for monitoring the progress of disease treatment or therapy in those patients undergoing treatment for a disease involving an VEGFR-2 modulator.

The biomarkers also serve as targets for the development of therapies for disease treatment. Such targets may be particularly applicable to treatment of cancer, such as, for example, hepatocellular carcinoma, colorectal cancer (CRC), NSCLC, and metastatic breast cancer.

Indeed, because these biomarkers are differentially expressed in sensitive and resistant cells, their expression patterns are correlated with relative intrinsic sensitivity of cells to treatment with VEGFR-2 modulators. Accordingly, the biomarkers highly expressed in resistant cells may serve as targets for the development of new therapies for the tumors which are resistant to VEGFR-2 modulators, particularly VEGFR-2 inhibitors. The level of biomarker protein and/or mRNA can be determined using methods well known to those skilled in the art. For example, quantification of protein can be carried out using methods such as ELISA, 2-dimensional SDS PAGE, Western blot, immunopreciptation, immunohistochemistry, fluorescence activated cell sorting (FACS), or flow cytometry. Quantification of mRNA can be carried out using methods such as PCR, array hybridization, Northern blot, in-situ hybridization, dot-blot, Taqman, or RNAse protection assay.

### Microarrays:

The invention also includes specialized microarrays, e.g., oligonucleotide microarrays or cDNA microarrays, comprising one or more biomarkers, showing expression profiles that correlate with either sensitivity or resistance to one or more VEGFR-2 modulators. Such microarrays can be employed in in vitro assays for assessing the expression level of the biomarkers in the test cells from tumor biopsies, and determining whether these test cells are likely to be resistant or sensitive to VEGFR-2 modulators. For example, a specialized microarray can be prepared using all the biomarkers, or subsets thereof, as described herein and shown in Tables 2-4. Cells from a tissue or organ biopsy can be isolated and exposed to one or more of the VEGFR-2 modulators. In one aspect, following application of nucleic acids isolated from both untreated and treated cells to one or more of the specialized microarrays, the pattern of gene expression of the tested cells can be determined and compared with that of the biomarker pattern from the control panel of cells used to create the biomarker set on the microarray. Based upon the gene expression pattern results from the cells that underwent testing, it can be determined if the cells show a resistant or a sensitive profile of gene expression. Whether or not the tested cells from a tissue or organ biopsy will respond to one or more of the VEGFR-2 modulators and the course of treatment or therapy can then be determined or evaluated based on the information gleaned from the results of the specialized microarray analysis.

### Antibodies:

The invention also includes antibodies, including polyclonal or monoclonal, directed against one or more of the polypeptide biomarkers. Such antibodies can be used in a variety of ways, for example, to purify, detect, and target the biomarkers of the invention, including both in vitro and in vivo diagnostic, detection, screening, and/or therapeutic methods.

### Kits:

The invention also includes kits for determining or predicting whether a patient would be susceptible or resistant to a treatment that comprises one or more VEGFR-2 modulators. The patient may have a cancer or tumor such as, for example, a breast cancer or tumor. Such kits would be useful in a clinical setting for use in testing a patient's biopsied tumor or cancer samples, for example, to determine or predict if the patient's tumor or cancer will be resistant or sensitive to a given treatment or therapy with an VEGFR-2 modulator. The kit comprises a suitable container that comprises: one or more microarrays, e.g., oligonucleotide microarrays or cDNA microarrays, that comprise those biomarkers that correlate with resistance and sensitivity to VEGFR-2 modulators, particularly VEGFR-2 inhibitors; one or more VEGFR-2 modulators for use in testing cells from patient tissue specimens or patient samples; and instructions for use. In addition, kits contemplated by the invention can further include, for example, reagents or materials for monitoring the expression of biomarkers of the invention at the level of mRNA or protein, using other techniques and systems practiced in the art such as, for example, RT-PCR assays, which employ primers designed on the basis of one or more of the biomarkers described herein, immunoassays, such as enzyme linked immunosorbent assays (ELISAs), immunoblotting, e.g., Western blots, or in situ hybridization, and the like, as further described herein.

### Application of Biomarkers and Biomarker Sets:

The biomarkers and biomarker sets may be used in different applications.

Biomarker sets can be built from any combination of biomarkers listed in Tables 2-4 to make predictions about the likely effect of any VEGFR-2 modulator in different biological systems. The various biomarkers and biomarkers sets described herein can be used, for example, as diagnostic or prognostic indicators in disease management, to predict how patients with cancer might respond to therapeutic intervention with compounds that modulate the VEGFR-2, and to predict how patients might respond to therapeutic intervention that modulates signaling through the entire VEGFR-2 regulatory pathway.

While the data described herein were generated in cell lines that are routinely used to screen and identify compounds that have potential utility for cancer therapy, the biomarkers have both diagnostic and prognostic value in other diseases areas in which signaling through VEGFR-2 or the VEGFR-2 pathway is of importance, e.g., in immunology, or in cancers or tumors in which cell signaling and/or proliferation controls have gone awry.

In accordance with the invention, cells from a patient tissue sample, e.g., a tumor or cancer biopsy, can be assayed to determine the expression pattern of one or more biomarkers prior to treatment with one or more VEGFR-2 modulators. Success or failure of a treatment can be determined based on the biomarker expression pattern of the cells from the test tissue (test cells), e.g., tumor or cancer biopsy, as being relatively similar or different from the expression pattern of a control set of the one or more biomarkers. Thus, if the test cells show a biomarker expression profile which corresponds to that of the biomarkers in the control panel of cells which are sensitive to the VEGFR-2 modulator, it is highly likely or predicted that the individual's cancer or tumor will respond favorably to treatment with the VEGFR-2 modulator. By contrast, if the test cells show a biomarker expression pattern corresponding to that of the biomarkers of the control panel of cells which are resistant to the VEGFR-2 modulator, it is highly likely or predicted that the individual's cancer or tumor will not respond to treatment with the VEGFR-2 modulator.

The invention also provides a method of monitoring the treatment of a patient having a disease treatable by one or more VEGFR-2 modulators. The isolated test cells from the patient's tissue sample, e.g., a tumor biopsy or blood sample, can be assayed to determine the expression pattern of one or more biomarkers before and after exposure to an VEGFR-2 modulator wherein, preferably, the VEGFR-2 modulator is an VEGFR-2 inhibitor. The resulting biomarker expression profile of the test cells before and after treatment is compared with that of one or more biomarkers as described and shown herein to be highly expressed in the control panel of cells that are either resistant or sensitive to an VEGFR-2 modulator. Thus, if a patient's response is sensitive to treatment by an VEGFR-2 modulator, based on correlation of the expression profile of the one or biomarkers, the patient's treatment prognosis can be qualified as favorable and treatment can continue. Also, if, after treatment with an VEGFR-2 modulator, the test cells don't show a change in the biomarker expression profile corresponding to the control panel of cells that are sensitive to the VEGFR-2 modulator, it can serve as an indicator that the current treatment should be modified, changed, or even discontinued. This monitoring process can indicate success or failure of a patient's treatment with an VEGFR-2 modulator and such monitoring processes can be repeated as necessary or desired.

The biomarkers of the invention can be used to predict an outcome prior to having any knowledge about a biological system. Essentially, a biomarker can be considered to be a statistical tool. Biomarkers are useful primarily in predicting the phenotype that is used to classify the biological system. In an embodiment of the invention, the goal of the prediction is to classify cancer cells as having an active or inactive VEGFR-2 pathway. Cancer cells with an inactive VEGFR-2 pathway can be considered resistant to treatment with an VEGFR-2 modulator.

### Examples

### Methods and Samples:

In the following examples, the compound [(1R), 2S]-2-Aminopropionic acid 2-[4-(4-fluoro-2-methyl-1H-indol-5-yloxy)-5-methylpyrrolo[2,1-f][1,2,4]triazin-6-yloxy]-1-methylethyl ester was used:

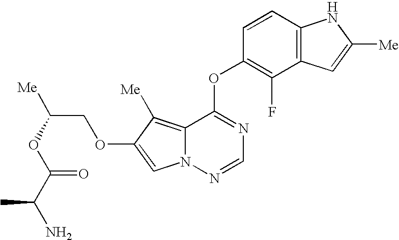

This compound is referred to herein as “compound 1.”

### Example 1

### Identification of Biomarkers

### Methods

Tumors were propagated in nude mice as subcutaneous (sc) implants using human GEO colon tumor cell lines. Tumors were maintained in nude mice by serial passage. Treated animals were checked daily for treatment related toxicity/mortality. Each group of animals was weighed before the initiation of treatment (Wt1) and then again following the last treatment dose (Wt2). The difference in body weight (Wt2-Wt1) provided an overt measure of treatment-related toxicity. No weight loss was observed in treated groups when compared to vehicle treated mice. Tumor response was determined by measurement of tumors with a caliper twice a week, until the tumors reached a predetermined target size. Animals that were distributed to various treatment and control groups during the study were eventually sacrificed to obtain tumor and blood samples for genomic and protein analysis (Table 1).

TABLE 1
Xenograft Groups and Treatments

| Group | |
|---|---|
| (6 mice per | |
| group) | Treatment |
| 1 | no treatment |
| 2 | bevacizumab (6 mg/kg) post 1 dose (2 hours) |
| 3 | compound 1 (100 mg/kg) post 1 dose (2 hours) |
| 4 | bevacizumab (6 mg/kg) q4 d × 5 (2 hours post last dose, |
| | day 17) |
| 5 | compound 1 (100 mg/kg) q1 d × 17 (2 hours post dose, |
| | day 17) |

Mouse GEO xenograft tumors, treated and non-treated, were surgically removed at appropriate time points and split into two halves. One half of the tumor was mixed in a vial of 10% formalin to be used for IHC and the other half was mixed in 1.0 ml of RNAlater™ RNA Stabilization Reagent (Ambion, Inc., Austin, Tex.) held at room temperature for 30-minutes then stored at −80° C. Total RNA was extracted from the tumor samples using the RNeasy Kit™ (Qiagen, Valencia Calif.). A least 1 μg total RNA with a 260/280 ratio >1.8 by spectrophotometry (DU640 UV, Beckman Coulter, Fullerton, Calif.) was required for transcriptional profiling. Briefly, the tumor samples were homogenized in a lysis-binding solution supplied by Qiagen. An equal volume of ethanol was mixed into each sample, the mixture was passed through the filter cartridges provided, and the rest of the extraction protocol was followed (Ambion). RNA yield and quality were assessed on a standard 1% agarose gel. A ratio 28 s/18 s RNA in the range of 1.5 to 2.5 indicated high quality RNA free of degradation. RNA samples were stored at −80° C.

Transcriptional profiling was performed on the RNA obtained from the GEO xenograft tumor samples. The Affymetrix GeneChip system (Affymetrix, Santa Clara, Calif.) was used for hybridization and scanning of the human U133A and mouse 430A arrays. Generation of cRNA followed a standard T7 amplification protocol. Total RNA was reverse-transcribed with SuperScript II™ (Gibco, Carlsbad, Calif.) in the presence of T7-(dT)24 primer to generate first strand cDNA. A second-strand cDNA synthesis was performed in the presence of DNA Polymerase I, DNA ligase, and RNase H (Gibco). The resulting double-stranded cDNA was blunt-ended using T4 DNA polymerase. This double-stranded cDNA was then transcribed into cRNA in the presence of biotin-ribonucleotides using the BioArray High Yield RNA transcript labeling kit (Enzo Life Sciences, Farmingdale, N.Y.). The amplified, biotin-labeled cRNA was purified using Qiagen RNeasy™ columns (Qiagen Sciences), quantified and fragmented at 94° C. for 35 minutes in the presence of fragmentation buffer (1×). Fragmented cRNA was hybridized to the Affymetrix U113A and 430A arrays overnight at 42° C. The arrays were then placed in the fluidics stations for staining and washing as recommended by Affymetrix protocols. The chips were scanned and raw intensity values were generated for each probe on the arrays.

### Statistical Analysis

20 HG-U133A2 chips were normalized by the RMA (Robust Multi-array Analysis) method as described by R. A. Irizarry et al., Nucleic Acids Res., February 15; 31(4):e15 (2003). The same normalization was done for the 20 Mouse430A2 chips.

### Identification of Differentially Expressed Genes

Identification of differentially expressed genes upon compound treatment was performed using Bioconductor limma package (Linear Models for Microarray Data), which has improved statistical power for experiments with small numbers of arrays. Differentially expressed genes were defined as: (i) FDR (false discovery rate) q value less than 0.05 when comparing specific compound treatment at single time point to the control (no treatment) at 0 time point and (ii) fold change bigger than 2 (log₂Foldchange>1 or <−1) when compared specific treatment to control.

Identification of Biomarkers that Specifically Affect Mouse Stromal Tissues

Identification of biomarkers that specifically affect mouse stroma tissues was focused on because these would give a general picture of how compound 1 or bevacizumab affects vasculature surrounding the tumor tissues. There were 113 probe sets representing 110 genes identified on Mouse430A2 chips as differentially expressed genes (FDR q value<0.05) upon compound 1 treatment at 17 Days. This list was reduced by selecting those with a fold change larger than 2 when compared to the control, resulting in 41 probe sets (39 genes). The 41 probe sets were mapped to HG-U133A2 chips whenever possible, and 20 genes whose expression changes on human chips were not significant (FDR q value>0.2) were selected.

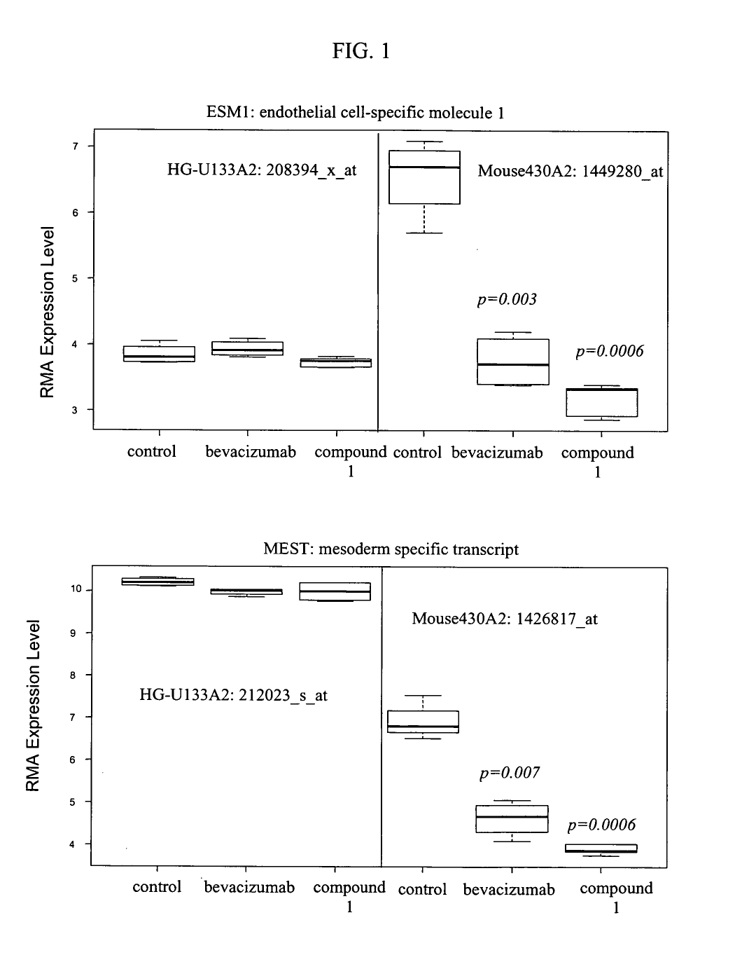

ESM1 (endothelial cell-specific molecule 1, also called endoncan), FST (follistatin), APH1A (anterior pharynx defective 1a homolog (C. elegans)) and INHBB (inhibin beta-B) are considered most interested ones and their expression patterns were plotted using R package. FIG. 1 shows the expression value of ESM1 in stromal tissue upon either compound 1 or bevacizumab treatment at 17 Days significantly decreases in comparison with the control. Importantly, even after 2 hours treatment, there is a decreasing trend in the ESM1 level by both agents. This result indicates that the expression of ESM1 at the mRNA level may respond to anti-angiogenic agents quickly and its level continues to decrease during the treatment. FIG. 1 also demonstrates that the impact of compound 1 on the ESM1 level is even more profound than that of bevacizumab at 17 days.

Table 2A provides the expression changes of significant genes in mouse stromal tissues. In Table 2A, genes are sorted by their fold change by compound 1 (com. 1) treatment: (i) category 1 (cat.): genes also changed significantly by bevacizumab (bev.) 17 days treatment; (ii) category 2: the extent of changed induced by bevacizumab and compound 1 treatment are significantly different; and (iii) category 3: the rest of genes (only changed by compound 1 treatment as defined above).

TABLE 2A
Genes that Specifically Affect Mouse Stromal Tissues Upon Compound 1
Treatment

| | | | | | difference | | |
|---|---|---|---|---|---|---|---|
| | | | | | between | difference | |
| | bev. | | com. 1 | | bev. and | between | |
| | log2 | | log2 | | com. 1 | bev. and | |
| | fold | bev. p | fold | com. 1 p | log2 fold | com. 1 p | |
| probe set ID | change | value | change | value | change | value | cat. |
| 1417851_at | 1.6837 | 0.9978 | 4.1105 | 0.0142 | 2.4268 | 0.1207 | 3 |
| 1419394_s_at | 1.9920 | 0.1651 | 3.2556 | 0.0049 | 1.2636 | 0.1941 | 3 |
| 1448756_at | 1.1326 | 0.9998 | 2.7363 | 0.0332 | 1.6037 | 0.1702 | 3 |
| 1417268_at | 0.2523 | 0.9998 | 2.5486 | 0.0021 | 2.2963 | 0.0050 | 2 |
| 1419149_at | 1.0236 | 0.3743 | 2.2328 | 0.0018 | 1.2092 | 0.0583 | 3 |
| 1428942_at | 0.6383 | 0.9998 | 2.1496 | 0.0185 | 1.5112 | 0.0804 | 3 |
| 1418457_at | 0.6694 | 0.9998 | 1.9297 | 0.0125 | 1.2603 | 0.0705 | 3 |
| 1416321_s_at | 0.6363 | 0.9998 | 1.6903 | 0.0471 | 1.0541 | 0.1710 | 3 |
| 1419665_a_at | 0.9402 | 0.5052 | 1.6533 | 0.0142 | 0.7131 | 0.2404 | 3 |
| 1427035_at | 0.6541 | 0.9998 | 1.5856 | 0.0221 | 0.9315 | 0.1444 | 3 |
| 1438211_s_at | 1.4915 | 0.0564 | 1.5264 | 0.0237 | 0.0349 | 0.9694 | 3 |
| 1419666_x_at | 0.8130 | 0.7015 | 1.4389 | 0.0283 | 0.6259 | 0.2922 | 3 |
| 1424302_at | 0.1770 | 0.9998 | 1.3842 | 0.0228 | 1.2072 | 0.0474 | 2 |
| 1425281_a_at | 0.9656 | 0.4785 | 1.3781 | 0.0428 | 0.4125 | 0.5152 | 3 |
| 1418456_a_at | 0.8855 | 0.4886 | 1.3591 | 0.0287 | 0.4737 | 0.4047 | 3 |
| 1419309_at | 0.1928 | 0.9998 | 1.3533 | 0.0192 | 1.1605 | 0.0452 | 2 |
| 1416125_at | 0.6576 | 0.7240 | 1.3503 | 0.0142 | 0.6927 | 0.1625 | 3 |
| 1426587_a_at | 0.6892 | 0.5271 | 1.3177 | 0.0126 | 0.6285 | 0.1660 | 3 |
| 1421034_a_at | 0.1847 | 0.9998 | 1.1326 | 0.0469 | 0.9479 | 0.0792 | 3 |
| 1421365_at | 0.1199 | 0.9998 | 1.0640 | 0.0333 | 0.9441 | 0.0543 | 3 |
| 1427844_a_at | 0.0299 | 0.9998 | 1.0382 | 0.0055 | 1.0083 | 0.0054 | 2 |
| 1438390_s_at | −0.2020 | 0.9998 | −1.0916 | 0.0256 | −0.8896 | 0.0623 | 3 |
| 1448688_at | −0.8705 | 0.3666 | −1.0948 | 0.0496 | −0.2243 | 0.6841 | 3 |
| 1448942_at | −1.0217 | 0.0174 | −1.1040 | 0.0073 | −0.0823 | 0.8478 | 1 |
| 1449147_at | −0.5207 | 0.9998 | −1.1088 | 0.0334 | −0.5882 | 0.2160 | 3 |
| 1449146_at | −0.8723 | 0.3660 | −1.1402 | 0.0412 | −0.2680 | 0.6154 | 3 |
| 1416211_a_at | −1.1978 | 0.0201 | −1.1586 | 0.0142 | 0.0393 | 0.9464 | 1 |
| 1451554_a_at | −0.1767 | 0.9998 | −1.1862 | 0.0006 | −1.0095 | 0.0038 | 2 |
| 1418181_at | −0.9981 | 0.1230 | −1.2198 | 0.0185 | −0.2217 | 0.6609 | 3 |
| 1417985_at | −0.3710 | 0.9998 | −1.2624 | 0.0073 | −0.8914 | 0.0447 | 2 |
| 1423516_a_at | −1.2432 | 0.0803 | −1.3889 | 0.0186 | −0.1457 | 0.8180 | 3 |
| 1459903_at | −0.8237 | 0.1138 | −1.4034 | 0.0018 | −0.5797 | 0.1341 | 3 |
| 1418142_at | −1.3672 | 0.1138 | −1.4093 | 0.0426 | −0.0421 | 0.9612 | 3 |
| 1428853_at | −0.9378 | 0.6726 | −1.5119 | 0.0465 | −0.5740 | 0.4008 | 3 |
| 1418499_a_at | −1.5445 | 0.0501 | −1.5662 | 0.0228 | −0.0217 | 0.9816 | 3 |
| 1451038_at | −0.7384 | 0.6529 | −1.6394 | 0.0081 | −0.9009 | 0.1044 | 3 |
| 1426858_at | −1.4395 | 0.1087 | −2.0687 | 0.0073 | −0.6292 | 0.3305 | 3 |
| 1420941_at | −2.3703 | 0.0488 | −2.1078 | 0.0454 | 0.2625 | 0.8144 | 1 |
| 1419589_at | −1.8871 | 0.0970 | −2.3397 | 0.0142 | −0.4527 | 0.6110 | 3 |
| 1423294_at | −2.2949 | 0.0070 | −2.9419 | 0.0006 | −0.6470 | 0.3568 | 1 |
| 1449280_at | −2.7929 | 0.0036 | −3.3695 | 0.0006 | −0.5766 | 0.4854 | 1 |

TABLE 2

| | NCBI Gene | | | DNA | | Protein | |
|---|---|---|---|---|---|---|---|
| | (LocusLink) | Gene | Probe | SEQ ID | DNA | SEQ ID | Protein |
| Entry | Title | Symbol | Set ID | NO: | Accession | NO: | Accession |
| 1 | N/A; | N/A; | 1417851_at | 1; 3; 4 | AF030636.1; | 2; 5 | AAC34603.1; |
| | chemokine | Cxcl13 | | | ENSMUST00000023840.3; | | NP_061354.1 |
| | (C—X—C motif) | | | | NM_018866.2 | | |
| | ligand 13 | | | | | | |
| 2 | S100 calcium | S100a8 | 1419394_s_at | 6 | NM_013650.2 | 7 | NP_038678.1 |
| | binding | | | | | | |
| | protein A8 | | | | | | |
| | (calgranulin | | | | | | |
| | A) | | | | | | |
| 3 | S100 calcium | S100a9 | 1448756_at | 8 | NM_009114.1 | 9 | NP_033140.1 |
| | binding | | | | | | |
| | protein A9 | | | | | | |
| | (calgranulin | | | | | | |
| | B) | | | | | | |
| 4 | CD14 antigen | Cd14 | 1417268_at | 10 | NM_009841.3 | 11 | NP_033971.1 |
| 5 | serine (or | Serpine 1 | 1419149_at | 12 | NM_008871.1 | 13 | NP_032897.1 |
| | cysteine) | | | | | | |
| | peptidase | | | | | | |
| | inhibitor, | | | | | | |
| | clade E, | | | | | | |
| | member 1 | | | | | | |
| 6 | Metallothionein 2 | Mt2 | 1428942_at | 14 | NM_008630.2 | 15 | NP_032656.1 |
| 7 | Chemokine | Cxcl14 | 1418457_at | 16 | NM_019568.2 | 17 | NP_062514.2 |
| | (C—X—C motif) | | | | | | |
| | ligand 14 | | | | | | |
| 8 | proline | Prelp | 1416321_s_at | 18 | NM_054077.2 | 19 | NP_473418.2 |
| | arginine-rich | | | | | | |
| | end leucine- | | | | | | |
| | rich repeat | | | | | | |
| 9 | Nuclear | Nupr1 | 1419665_a_at | 20 | NM_019738.1 | 21 | NP_062712.1 |
| | protein 1 | | | | | | |
| 10 | solute carrier | Slc39a | 1427035_at | 22; 24; | AK122197.1; | 23; 27 | BAC65479.2; |
| | family 39 | | | 25; 26 | BB399837.2; | | NP_659057.2 |
| | (zinc | | | | BC030883.1; | | |
| | transporter), | | | | NM_144808.4 | | |
| | member 14 | | | | | | |
| 11 | N/A; D site | N/A; | 1438211_s_at | 28; 30; | AK140243.1; | 29; 33 | BAE24294.1; |
| | albumin | Dbp | | 31; 32 | BB550183.1; | | NP_058670.1 |
| | promoter | | | | BC064094.1; | | |
| | binding | | | | NM_016974.1 | | |
| | protein | | | | | | |
| 12 | Nuclear | Nupr1 | 1419666_x_at | 20 | NM_019738.1 | 21 | NP_062712.1 |
| | protein 1 | | | | | | |
| 13 | hypothetical | N/A; | 1424302_at | 34; 36; | NM_011095.1; | 35; 37 | NP_035225.1; |
| | LOC619608; | Lilrb3 | | 38 | XM_974271.1; | | XP_979365.1 |
| | Leukocyte | | | | XR_002514.1 | | |
| | immunoglobulin- | | | | | | |
| | like | | | | | | |
| | receptor, | | | | | | |
| | subfamily B | | | | | | |
| | (With TM and | | | | | | |
| | ITIM | | | | | | |
| | domains), | | | | | | |
| | member 3 | | | | | | |
| 14 | TSC22 domain | Tsc22d3 | 1425281_a_at | 39 | NM_010286.3 | 40 | NP_034416.3 |
| | family 3 | | | | | | |
| 15 | Chemokine | Cxcl14 | 1418456_a_at | 16 | NM_019568.2 | 17 | NP_062514.2 |
| | (C—X—C motif) | | | | | | |
| | ligand 14 | | | | | | |
| 16 | PODOPLANIN | Pdpn | 1419309_at | 41 | NM_010329.2 | 42 | NP_034459.2 |
| 17 | FK506 | Fkbp5 | 1416125_at | 43 | NM_010220.2 | 44 | NP_034350.1 |
| | binding | | | | | | |
| | protein 5 | | | | | | |
| 18 | Signal | Stat3 | 1426587_a_at | 45; 47; | NM_011486.4; | 46; 48; | NP_035616.1; |
| | transducer and | | | 49 | NM_213659.2; | 50 | NP_998824.1; |
| | activator of | | | | NM_213660.2 | | NP_998825.1 |
| | transcription 3 | | | | | | |
| 19 | Interleukin 4 | Il4ra | 1421034_a_at | 51 | NM_001008700.3 | 52 | NP_001008700.1 |
| | receptor, alpha | | | | | | |
| 20 | Follistatin | Fst | 1421365_at | 53 | NM_008046.2 | 54 | NP_032072.1 |
| 21 | CCAAT/enhancer | Cebpb | 1427844_a_at | 55 | NM_009883.1 | 56 | NP_034013.1 |
| | binding | | | | | | |
| | protein | | | | | | |
| | (C/EBP), beta | | | | | | |
| 22 | Pituitary | Pttg1 | 1438390_s_at | 57 | NM_013917.1 | 58 | NP_038945.1 |
| | tumor- | | | | | | |
| | transforming 1 | | | | | | |
| 23 | Podocalyxin- | Podxl | 1448688_at | 59 | NM_013723.2 | 60 | NP_038751.2 |
| | like | | | | | | |
| 24 | Guanine | Gng11 | 1448942_at | 61 | NM_025331.2 | 62 | NP_079607.1 |
| | nucleotide | | | | | | |
| | binding | | | | | | |
| | protein (G | | | | | | |
| | protein), | | | | | | |
| | gamma 11 | | | | | | |
| 25 | carbohydrate | Chst1 | 1449147_at | 63 | NM_023850.1 | 64 | NP_076339.1 |
| | (keratan | | | | | | |
| | sulfate Gal-6) | | | | | | |
| | sulfotransferase 1 | | | | | | |
| 26 | Notch gene | Notch4 | 1449146_at | 65 | NM_010929.2 | 66 | NP_035059.2 |
| | homolog 4 | | | | | | |
| | (Drosophila) | | | | | | |
| 27 | Pleiotrophin | Ptn | 1416211_a_at | 67 | NM_008973.2 | 68 | NP_032999.1 |
| 28 | N/A; anterior | N/A; | 1451554_a_at | 69; 71; | BC012406.1; | 70; 73 | AAH12406.1; |
| | pharynx | Aph1a | | 72 | ENSMUST00000015894.4; | | NP_666216.1 |
| | defective 1a | | | | NM_146104.1 | | |
| | homolog (C. elegans) | | | | | | |
| | 94.4; | | | | | | |
| 29 | protein | pPtp4a3 | 1418181_at | 74 | NM_008975.2 | 75 | NP_033001.2 |
| | tyrosine | | | | | | |
| | phosphatase | | | | | | |
| | 4a3 | | | | | | |
| 30 | Notch- | Nrarp | 1417985_at | 76 | NM_025980.2 | 77 | NP_080256.2 |
| | regulated | | | | | | |
| | ankyrin repeat | | | | | | |
| | protein | | | | | | |
| 31 | Nidogen 2 | Nid2 | 1423516_a_at | 78 | NM_008695.2 | 79 | NP_032721.2 |
| 32 | sema domain, | Sema7a | 1459903_at | 80 | NM_011352.2 | 81 | NP_035482.1 |
| | immunoglobulin | | | | | | |
| | domain (Ig), | | | | | | |
| | and GPI | | | | | | |
| | membrane | | | | | | |
| | anchor, | | | | | | |
| | (semaphorin) | | | | | | |
| | 7A | | | | | | |
| 33 | Potassium | Kcnj8 | 1418142_at | 82 | NM_008428.2 | 83 | NP_032454.1 |
| | inwardly- | | | | | | |
| | rectifying | | | | | | |
| | channel, | | | | | | |
| | subfamily J, | | | | | | |
| | member 8 | | | | | | |
| 34 | N/A; Patched | N/A; | 1428853_at | 84; 85 | BG071079.2; | | |
| | homolog 1 | Ptch1 | | | AK147626.1 | | |
| | {POOR HIT | (63%) | | | | | |
| | 63%} | | | | | | |
| 35 | potassium | Kcne3 | 1418499_a_at | 86 | NM_020574.3 | 87 | NP_065599.1 |
| | voltage-gated | | | | | | |
| | channel, Isk- | | | | | | |
| | related | | | | | | |
| | subfamily, | | | | | | |
| | gene 3 | | | | | | |
| 36 | APELIN | Apln | 1451038_at | 88 | NM_013912.3 | 89 | NP_038940.1 |
| 37 | inhibin beta-B | Inhbb | 1426858_at | 90; 92 | NM_008381.1; | 91; 93 | NP_032407.1; |
| | | | | | XM_984243.1 | | XP_989337.1 |
| 38 | Regulator of | Rgs5 | 1420941_at | 94 | NM_009063.2 | 95 | NP_033089.2 |
| | G-protein | | | | | | |
| | signaling 5 | | | | | | |
| 39 | CD93 antigen | Cd93 | 1419589_at | 96 | NM_010740.2 | 97 | NP_034870.1 |
| 40 | Mesoderm | Mest | 1423294_at | 98 | NM_008590.1 | 99 | NP_032616.1 |
| | specific | | | | | | |
| | transcript | | | | | | |
| 41 | similar to | N/A; | 1449280_at | 100; | NM_023612.3; | 101; 103 | NP_076101.1; |
| | Endothelial | Esm1 | | 102 | XM_906879.2 | | XP_911972.1 |
| | cell-specific | | | | | | |
| | molecule 1 | | | | | | |
| | precursor | | | | | | |
| | (ESM-1 | | | | | | |
| | secretory | | | | | | |
| | protein) | | | | | | |
| | (ESM-1); | | | | | | |
| | endothelial | | | | | | |
| | cell-specific | | | | | | |
| | molecule 1 | | | | | | |

### Biological Function of the Significant Genes

### ESM1 (Endothelial Cell-Specific Molecule 1)

ESM1 is a soluble proteoglycan of 50 kDa, constituted of a mature polypeptide of 165 amino acids, which is expressed by the vascular endothelium and has been found freely circulating in the bloodstream of healthy subjects. Experimental evidence shows ESM1 is a key player in the regulation of major processes such as cell adhesion, in inflammatory disorders and tumor progression. Inflammatory cytokines such as TNF-alpha, and pro-angiogenic growth factors such as VEGF, FGF-2 and HGF/SF, strongly increased the expression, synthesis or the secretion of endocan by human endothelial cells. In tumor patients, the ESM1 level is elevated in serum as measured by enzyme-linked immunoassay. Recently, the mRNA level of ESM1 has also been recognized as being one of the most significant molecular signatures of a bad prognosis in several types of cancer including lung cancer. ESM1 has been suggested as a biomarker in monitoring anti-angiogenesis in cancer therapy. (S. Sarrazin et al., Biochim Biophys Acta., 1765:25-37 (2006); A. Scherpereel, et al., Cancer Research, 63:6084-6089 (2003))

### MEST: Mesoderm Specific Transcript (Also Known as PEG1, Paternally Expressed Gene 1. FIG. 1)

Human mesoderm-specific transcript (MEST) is an imprinted gene expressed from the paternal allele and located on chromosome 7q32. In a recent study, MEST was found to be highly differentially expressed (up 8 fold) in invasive cervical carcinoma (CVX) compared to normal cervical keratinocytes (NCK). Frequent loss of imprinting (LOI) of PEG1/MEST has been implicated in the development of breast carcinoma, lung adenocarcinomas and colon cancer. Although the function of MEST gene product is unknown, the putative protein shares homology with the α/β hydrolase family, which also includes the lysosomal enxyme cathepsin A. This may suggest a possible role of MEST in the degradation of the extracellular matrix in the invasive state of cervical tumors. (A. Santin et al., Virology, January 20; 331 (2):269-91 (2005)).

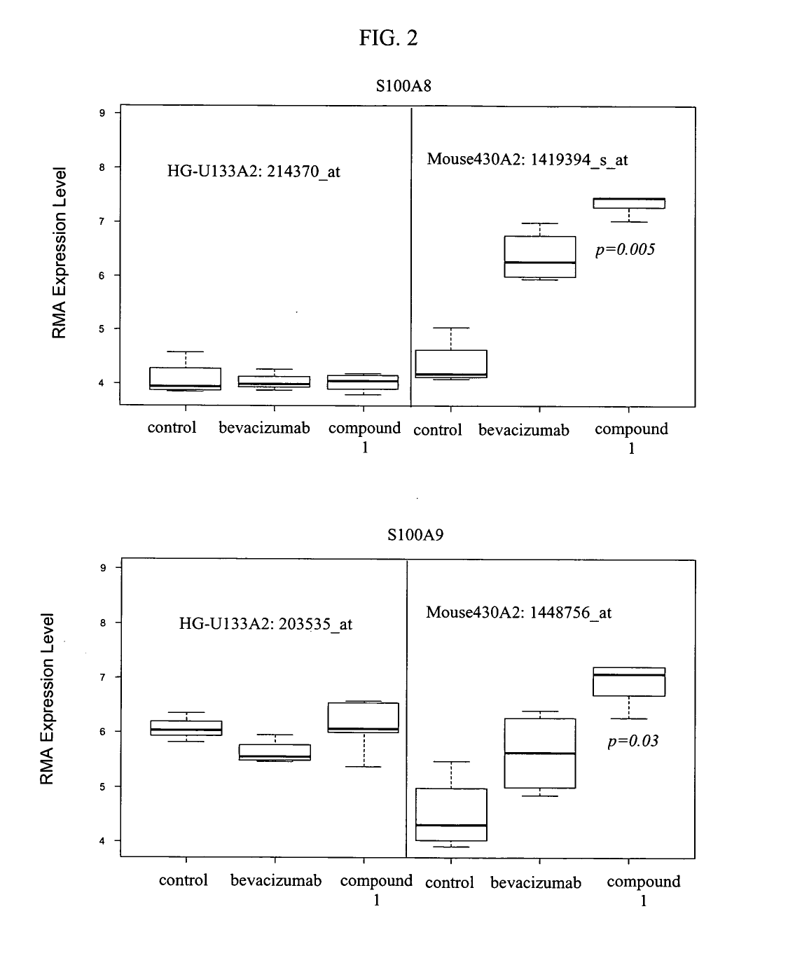

### S100A8/S100A9 (MRP8/MRP14. FIG. 2)

S100A8 and S100A9 are members of the S100 family of Ca2+-binding proteins and S100A8/A9 is the most abundant naturally occurring S100 heterodimer. S100A8 and S100A9 are considered important pro-inflammatory mediators in acute and chronic inflammation. They are expressed by keratinocytes and inflammatory cells in human/murine wounds and by appropriately activated macrophages, endothelial cells, epithelial cells, and keratinocytes in vitro. Strong S100A8 and S100A9 up-regulation was found in breast, lung, gastric, colorectal, pancreatic and prostate cancer. They are involved in various biological functions including cytoskeletal rearrangements, cell migration, arachidonic acid metabolism, and regulation of neutrophilic NADPH-oxidase. S100A8/A9 acts as a secreted protein complex and is the ligand for RAGE (receptor of advanced glycation end products). Co-expression of S100 proteins with RAGE (also known as AGER: advanced glycosylation end product-specific receptor) has been found in different types of tumor tissues. There are multiple RAGE downstream pathways including MAP kinases, PI3K, Ras and NF-κB. (C. Gebhardt et al., Biochem Pharmacol., November 30; 72(11):1622-31 (2006)).

The S100A8/S100A9 complex is released specifically at inflammatory sites and leads to increased serum levels in correlation with the degree of inflammation, indicating an extracellular role of these molecules in inflammatory processes. There are multiple findings indicating interactions between S100A8/S100A9 complex and endothelial cells. The protein complex is deposited on endothelia and bind specifically to human microvascular endothelial cells (HMECs). This complex also binds to carboxylated N-glycans expressed on inflammatory activated endothelial cells. The S100A8/S100A9 complex can increase the binding capacity of CD11b-CD18 on leukocytes to intracellular adhesion molecule-1 (ICAM-1) on endothelium. The S100A8/S100A9 complex directly induces a distinct inflammatory, thrombogenic response in HMECs and is characterized by the induction of proinflammatory chemokines and adhesion molecules and by increased vascular permeability. (D. Viemann et al., Blood, April 1; 105(7):2955-62. 2005))

In the current study, expression levels of S100A8 and S100A9 were both up-regulated in mouse stroma tissue upon compound 1 treatment at 17 days time point. Interestingly, the expression level of RAGE was also up-regulated in human xenograft tissue under the same treatment conditions (up 1.9 fold, FDR q=0.002). A reasonable hypothesis would be treatment of compound 1 promotes an acute inflammatory response in the mouse stroma tissue surrounding xenografts, as indicated by the elevated levels of S100A8 and S100A9. An acute inflammatory response against tumor xenografts is also supported by elevated CD14 (a surface molecule for monocytic cells) expression upon compound 1 treatment. Elevated ligand expression promotes RAGE expression levels in xenografts and subsequently activates signal pathways downstream RAGE, which normally promote tumor formation.

S100A8 mRNA expression in murine fibroblasts can also be induced by FGF-2 (F. Rahimi et al., FEBS J., June; 272(11):2811-27 (2005)). Even though FGF-2 levels didn't change much in the current experiment due to low expression, FGF-8, FGF-18, FGF-22 and FGFR-2 levels were all elevated in tumor xenografts under compound 1 treatment after 17 days.

Identification of Genes that Specifically Affect Tumor Tissues by both Compound 1 and Bevacizumab

Table 3A provides genes differentially expressed (FDR q value<0.05; Fold change>2) on HG-U133A2 chips upon treatment of compound 1 or bevacizumab. Genes are sorted by their fold change by compound 1 treatment. Two genes in category 1 (CDC27 and CSPG6) also have significant differences when comparing the extent of changes by bevacizumab and compound 1 treatment.

TABLE 3A
Genes That Specifically Affect Tumor Tissues by Both Compound 1 and
Bevacizumab

| | | | | | difference | | |
|---|---|---|---|---|---|---|---|
| | bev. | | com. 1 | | between | difference | |
| | log2 | | log2 | | bev. com. 1 | between | |
| | fold | bev. p | fold | com. 1 | log2 fold | bev. com. 1 | |
| probe set ID | change | value | change | p value | change | p value | cat. |
| 211074_at | 2.1465 | 0.0039 | 2.2582 | 0.0007 | 0.1118 | 0.8556 | 2 |
| 201324_at | 1.3329 | 0.0328 | 1.9803 | 0.0006 | 0.6474 | 0.1423 | 2 |
| 201061_s_at | 1.0760 | 0.0230 | 1.4890 | 0.0007 | 0.4131 | 0.2196 | 2 |
| 205483_s_at | 1.2276 | 0.0173 | 1.4417 | 0.0011 | 0.2141 | 0.5876 | 2 |
| 207847_s_at | 1.4949 | 0.0118 | 1.3284 | 0.0041 | −0.1666 | 0.7245 | 2 |
| 209921_at | 1.0484 | 0.0251 | 1.2819 | 0.0012 | 0.2336 | 0.5012 | 2 |
| 207158_at | 0.8843 | 0.0369 | 1.2325 | 0.0009 | 0.3481 | 0.2452 | 2 |
| 208436_s_at | 1.1961 | 0.0030 | 1.2092 | 0.0007 | 0.0130 | 0.9719 | 2 |
| 208966_x_at | 1.4436 | 0.0117 | 1.1920 | 0.0063 | −0.2516 | 0.5631 | 2 |
| 202074_s_at | 1.0289 | 0.0093 | 1.1915 | 0.0008 | 0.1626 | 0.5901 | 2 |
| 213693_s_at | 1.2759 | 0.0044 | 1.1479 | 0.0019 | −0.1280 | 0.7193 | 2 |
| 204981_at | 0.9087 | 0.0029 | 1.1407 | 0.0003 | 0.2320 | 0.2777 | 2 |
| 201482_at | 1.1017 | 0.0386 | 1.1383 | 0.0052 | 0.0366 | 0.9385 | 2 |
| 207574_s_at | 0.8582 | 0.0042 | 1.0736 | 0.0004 | 0.2154 | 0.3184 | 2 |
| 206332_s_at | 1.4322 | 0.0244 | 1.0437 | 0.0227 | −0.3885 | 0.4018 | 2 |
| 200696_s_at | 1.0650 | 0.0065 | 1.0382 | 0.0016 | −0.0268 | 0.9391 | 2 |
| 209835_x_at | 1.2007 | 0.0347 | 1.0335 | 0.0131 | −0.1672 | 0.7089 | 2 |
| 221705_s_at | −1.4467 | 0.0014 | −1.0156 | 0.0033 | 0.4311 | 0.1652 | 2 |
| 213971_s_at | −1.0343 | 0.0040 | −1.0206 | 0.0010 | 0.0137 | 0.9668 | 2 |
| 201996_s_at | −0.9345 | 0.0019 | −1.0522 | 0.0004 | −0.1177 | 0.5993 | 2 |
| 212079_s_at | −0.6658 | 0.0388 | −1.0837 | 0.0005 | −0.4179 | 0.0699 | 2 |
| 220232_at | −1.0538 | 0.0206 | −1.0910 | 0.0028 | −0.0372 | 0.9264 | 2 |
| 212926_at | −1.1631 | 0.0031 | −1.0912 | 0.0011 | 0.0719 | 0.8197 | 2 |
| 212030_at | −0.7839 | 0.0276 | −1.1182 | 0.0007 | −0.3343 | 0.1830 | 2 |
| 201927_s_at | −1.3798 | 0.0001 | −1.1245 | 0.0005 | 0.2553 | 0.2778 | 2 |
| 217878_s_at | −2.4757 | 0.0000 | −1.1781 | 0.0008 | 1.2976 | 0.0010 | 1 |
| 209258_s_at | −2.2926 | 0.0001 | −1.1992 | 0.0044 | 1.0934 | 0.0105 | 1 |
| 214537_at | −1.5073 | 0.0023 | −1.2426 | 0.0019 | 0.2647 | 0.4601 | 2 |
| 220295_x_at | −0.8566 | 0.0333 | −1.3621 | 0.0005 | −0.5055 | 0.0784 | 2 |

TABLE 3

| | | | | DNA | | | |
|---|---|---|---|---|---|---|---|
| | NCBI Gene | | | SEQ | | Protein | |
| | (LocusLink) | Gene | Probe | ID | DNA | SEQ | Protein |
| Entry | Title | Symbol | Set ID | NO: | Accession | ID NO: | Accession |
| 1 | folate | FOLR1 | 211074_at | 104 | AF000381.2 | 105 | AAB81938.2 |
| | receptor 1 | (27%) | | | | | |
| | (adult) | | | | | | |
| | {POOR HIT | | | | | | |
| | 27%} | | | | | | |
| 2 | Epithelial | EMP1 | 201324_at | 106 | NM_001423.1 | 107 | NP_001414.1 |
| | membrane | | | | | | |
| | protein 1 | | | | | | |
| 3 | Stomatin | STOM | 201061_s_at | 108; | NM_004099.4; | 109; | NP_004090.4; |
| | | | | 110 | NM_198194.1 | 111 | NP_937837.1 |
| 4 | ISG15 | ISG15 | 205483_s_at | 112 | NM_005101.1 | 113 | NP_005092.1 |
| | ubiquitin-like | | | | | | |
| | modifier | | | | | | |
| 5 | Mucin 1, cell | MUC1 | 207847_s_at | 114; | NM_001018016.1; | 115; | NP_001018016.1; |
| | surface | | | 116; | NM_001018017.1; | 117; | NP_001018017.1; |
| | associated | | | 118 | NM_002456.4 | 119 | NP_002447.4 |
| 6 | solute carrier | SLC7A11 | 209921_at | 120; | AB040875.1; | 121; | BAB40574.1; |
| | family 7, | | | 122 | NM_014331.3 | 123 | NP_055146.1 |
| | (cationic | | | | | | |
| | amino acid | | | | | | |
| | transporter, | | | | | | |
| | y+ system) | | | | | | |
| | member 11 | | | | | | |
| 7 | apolipoprotein | APOBEC1 | 207158_at | 124 | NM_001644.3 | 125 | NP_001635.2 |
| | B mRNA | | | | | | |
| | editing | | | | | | |
| | enzyme, | | | | | | |
| | catalytic | | | | | | |
| | polypeptide 1 | | | | | | |
| 8 | Interferon | IRF7 | 208436_s_at | 126; | NM_001572.3; | 127; | NP_001563.2; |
| | regulatory | | | 128; | NM_004029.2; | 129; | NP_004020.1; |
| | factor 7 | | | 130 | NM_004031.2 | 131 | NP_004022.2 |
| 9 | Interferon, | IFI16 | 208966_x_at | 132 | AF208043.1 | 133 | AAF20997.1 |
| | gamma- | (58%) | | | | | |
| | inducible | | | | | | |
| | protein 16 | | | | | | |
| | {POOR HIT | | | | | | |
| | 58%} | | | | | | |
| 10 | Optineurin | OPTN | 202074_s_at | 134; | NM_001008211.1; | 135; | NP_001008212.1; |
| | | | | 136; | NM_001008212.1; | 137; | NP_001008213.1; |
| | | | | 138; | NM_001008213.1; | 139; | NP_001008214.1; |
| | | | | 140 | NM_021980.4 | 141 | NP_068815.2 |
| 11 | Mucin 1, cell | MUC1 | 213693_s_at | 114; | NM_001018016.1; | 115; | NP_001018016.1; |
| | surface | | | 116; | NM_001018017.1; | 117; | NP_001018017.1; |
| | associated | | | 118 | NM_002456.4 | 119 | NP_002447.4 |
| 12 | solute carrier | SLC22A18 | 204981_at | 142 | NM_002555.3 | 143 | NP_002546.2 |
| | family 22 | | | | | | |
| | (organic | | | | | | |
| | cation | | | | | | |
| | transporter), | | | | | | |
| | member 18 | | | | | | |
| 13 | Quiescin Q6 | QSCN6 | 201482_at | 144; | NM_001004128.2; | 145; | NP_001004128.1; |
| | | | | 146 | NM_002826.4 | 147 | NP_002817.2 |
| 14 | Growth arrest | GADD45B | 207574_s_at | 148 | NM_015675.2 | 149 | NP_056490.2 |
| | and DNA- | | | | | | |
| | damage- | | | | | | |
| | inducible, | | | | | | |
| | beta | | | | | | |
| 15 | Interferon, | IFI16 | 206332_s_at | 150 | NM_005531.2 | 151 | NP_005522.2 |
| | gamma- | | | | | | |
| | inducible | | | | | | |
| | protein 16 | | | | | | |
| 16 | gelsolin | GSN | 200696_s_at | 152; | NM_000177.4; | 153; | NP_000168.1; |
| | (amyloidosis, | | | 154 | NM_198252.2 | 155 | NP_937895.1 |
| | Finnish type) | | | | | | |
| 17 | CD44 | CD44 | 209835_x_at | 156; | NM_000610.3; | 157; | NP_000601.3; |
| | molecule | | | 158; | NM_001001389.1; | 159; | NP_001001389.1; |
| | (Indian blood | | | 160 | NM_001001390.1 | 161 | NP_001001390.1 |
| | group) | | | | | | |
| 18 | Suppressor of | SIKE | 221705_s_at | 162 | NM_025073.1 | 163 | NP_079349.1 |
| | IKK epsilon | (*) | | | | | |
| 19 | suppressor of | SUZ12 | 213971_s_at | 164 | NM_015355.1 | 165 | NP_056170.1 |
| | zeste 12 | | | | | | |
| | homolog | | | | | | |
| | (Drosophila) | | | | | | |
| 20 | spen | SPEN | 201996_s_at | 166 | NM_015001.2 | 167 | NP_055816.2 |
| | homolog, | | | | | | |
| | transcriptional | | | | | | |
| | regulator | | | | | | |
| | (Drosophila) | | | | | | |
| 21 | myeloid/lymphoid | MLL | 212079_s_at | 168 | NM_005933.2 | 169 | NP_005924.2 |
| | or | | | | | | |
| | mixed- | | | | | | |
| | lineage | | | | | | |
| | leukemia | | | | | | |
| | (trithorax | | | | | | |
| | homolog, | | | | | | |
| | Drosophila) | | | | | | |
| 22 | stearoyl-CoA | SCD5 | 220232_at | 170 | NM_024906.1 | 171 | NP_079182.1 |
| | desaturase 5 | | | | | | |
| 23 | Structural | SMC5 | 212926_at | 172 | NM_015110.1 | 173 | NP_055925.1 |
| | maintenance | | | | | | |
| | of | | | | | | |
| | chromosomes 5 | | | | | | |
| 24 | RNA binding | RBM25 | 212030_at | 174 | NM_021239.1 | 175 | NP_067062.1 |
| | motif protein | | | | | | |
| | 25 | | | | | | |
| 25 | Plakophilin 4 | PKP4 | 201927_s_at | 176; | NM_001005476.1; | 177; | NP_001005476.1; |
| | | | | 178 | NM_003628.3 | 179 | NP_003619.2 |
| 26 | cell division | CDC27 | 217878_s_at | 180 | NM_001256.2 | 181 | NP_001247.2 |
| | cycle 27 | | | | | | |
| | homolog (S. cerevisiae) | | | | | | |
| 27 | Structural | SMC3 | 209258_s_at | 182 | NM_005445.3 | 183 | NP_005436.1 |
| | maintenance | | | | | | |
| | of | | | | | | |
| | chromosomes 3 | | | | | | |
| 28 | histone | HIST1H1D | 214537_at | 184 | NM_005320.2 | 185 | NP_005311.1 |
| | cluster 1, H1d | | | | | | |
| 29 | DEP domain | DEPDC1 | 220295_x_at | 186 | NM_017779.3 | 187 | NP_060249.2 |
| | containing 1 | | | | | | |

### Biological Functions of Significant Genes

### EMP1 (Epithelial Membrane Protein 1. Also Known as TMP: Tumor Membrane Protein)

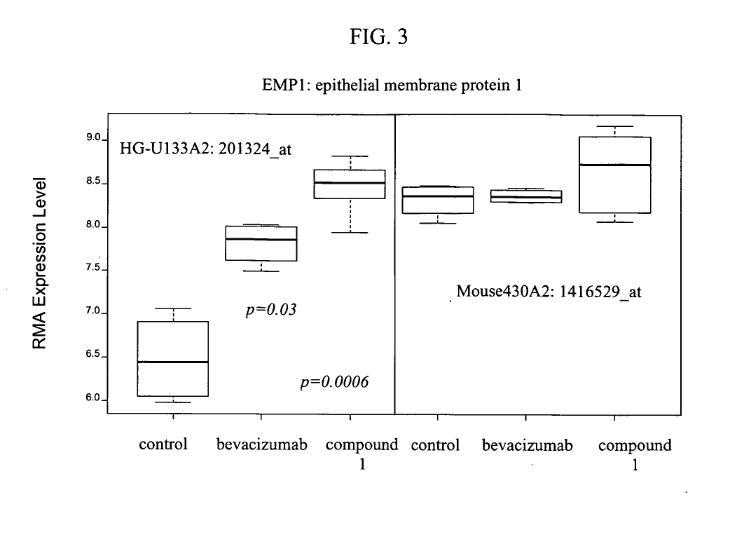

It is noted that EMP-1 expression is 6 fold down-regulated in esophageal cancer compared to normal tissue. It is highly up-regulated during squamous cell differentiation and has a role in tumorigenesis in certain tumors. Overexpression of human EMP-1 gene can inhibit the proliferation of certain tumor cells with S phase arrested and G1 phase prolonged (H. Wang et al., World J Gastroenterol., March; 9(3):392-8 (2003)). In our experiment, EMP1 was up-regulated 2.5 fold by bevacizumab and 3.9 fold by compound 1, both with significant FDR q values. This indicates a potential anti-proliferation effect by both compounds (FIG. 3).

EMP1 has been identified as a clinical biomarker whose expression was correlated with lack of complee or partial response to gefitinib in lung cancer patient samples as well as clinical progression to secondary gefitinib resistance. Its expression was also correlated with acquisition of gefitinib resistance in a mouse xenograft model. Such correlations are independent of gefitinib-sensitizing EGFR somatic mutations (A. Jain et al., P.N.A.S. USA., August 16; 102(33):11858-63 (2005)).

Identification of Genes that Specifically Affect Tumor Tissues Only by Compound 1

Table 4A provides the top 40 genes that are differentially expressed on HG-U133A2 chips (FDR<0.05, fold change>2) specifically upon compound 1 treatment at 17 Days. These markers were not changed by bevacizumab treatment at 17 Days (FDR>0.2). Genes are sorted by their fold change by compound 1 treatment. Three genes (DAPK1, HIG2, AKAP7) changed in opposite directions by bevacizumab and compound 1 treatment.

TABLE 4A
Genes that Specifically Affect Tumor Tissues only by Compound 1

| | bev. 17 | | com. 1 | | difference | | |
|---|---|---|---|---|---|---|---|
| | days | | 17 days | | bev. com. 1 | difference | |
| | log2 | bev. 17 | log2 | com. 1 | 17 days | bev. com. 1 | |
| | fold | days p | fold | 17 days | log2 fold | 17 days p | |
| probe set ID | change | value | change | p value | change | value | cat. |
| 213350_at | 0.1890 | 0.9504 | 2.3542 | 0.0134 | 2.1652 | 0.0247 | 2 |
| 212190_at | 0.2629 | 0.7959 | 2.1748 | 0.0003 | 1.9119 | 0.0010 | 2 |
| 206576_s_at | 0.2751 | 0.8710 | 2.1443 | 0.0024 | 1.8692 | 0.0076 | 2 |
| 212044_s_at | 0.5848 | 0.6680 | 2.1271 | 0.0027 | 1.5424 | 0.0200 | 2 |
| 204259_at | 1.0356 | 0.2352 | 1.9635 | 0.0019 | 0.9280 | 0.0895 | 2 |
| 211889_x_at | 0.1466 | 0.9250 | 1.8274 | 0.0017 | 1.6807 | 0.0045 | 2 |
| 216246_at | 0.4582 | 0.7420 | 1.7816 | 0.0054 | 1.3234 | 0.0317 | 2 |
| 209160_at | 0.1178 | 0.9108 | 1.7767 | 0.0004 | 1.6589 | 0.0010 | 2 |
| 217685_at | 0.2692 | 0.7800 | 1.7533 | 0.0006 | 1.4842 | 0.0026 | 2 |
| 209498_at | 0.8324 | 0.2326 | 1.7335 | 0.0010 | 0.9011 | 0.0434 | 2 |
| 208596_s_at | 0.2725 | 0.7014 | 1.7249 | 0.0003 | 1.4524 | 0.0012 | 2 |
| 201926_s_at | 0.6294 | 0.2737 | 1.6951 | 0.0005 | 1.0656 | 0.0074 | 2 |
| 220798_x_at | 0.1492 | 0.9067 | 1.6937 | 0.0011 | 1.5445 | 0.0035 | 2 |
| 209365_s_at | 0.6082 | 0.2952 | 1.6917 | 0.0005 | 1.0835 | 0.0066 | 2 |
| 215177_s_at | 0.3586 | 0.8503 | 1.6914 | 0.0195 | 1.3328 | 0.0639 | 2 |
| 220593_s_at | 0.3487 | 0.6811 | 1.6867 | 0.0007 | 1.3380 | 0.0039 | 2 |
| 218002_s_at | 0.1505 | 0.8972 | 1.6801 | 0.0008 | 1.5296 | 0.0026 | 2 |
| 204803_s_at | 0.2621 | 0.7871 | 1.6761 | 0.0008 | 1.4140 | 0.0033 | 2 |
| 220894_x_at | 0.3548 | 0.6506 | 1.6556 | 0.0006 | 1.3008 | 0.0037 | 2 |
| 203191_at | 0.1885 | 0.8641 | 1.6303 | 0.0010 | 1.4418 | 0.0035 | 2 |
| 203139_at | 0.2871 | 0.7611 | −1.2674 | 0.0036 | −1.5545 | 0.0021 | 1 |
| 219703_at | −0.6643 | 0.3329 | −1.2748 | 0.0041 | −0.6105 | 0.1313 | 2 |
| 219644_at | −0.5085 | 0.4415 | −1.2799 | 0.0023 | −0.7714 | 0.0402 | 2 |
| 204641_at | −0.4842 | 0.4573 | −1.3042 | 0.0018 | −0.8200 | 0.0276 | 2 |
| 218108_at | −0.1047 | 0.9311 | −1.3072 | 0.0028 | −1.2024 | 0.0066 | 2 |
| 206102_at | −0.5989 | 0.4280 | −1.3204 | 0.0042 | −0.7215 | 0.0888 | 2 |
| 209642_at | −0.6627 | 0.4330 | −1.3306 | 0.0077 | −0.6679 | 0.1551 | 2 |
| 218979_at | −0.1925 | 0.8562 | −1.3416 | 0.0027 | −1.1491 | 0.0091 | 2 |
| 218507_at | 0.0933 | 0.8945 | −1.3761 | 0.0003 | −1.4694 | 0.0002 | 1 |
| 203764_at | −0.3963 | 0.5346 | −1.3799 | 0.0009 | −0.9836 | 0.0085 | 2 |
| 205283_at | −0.2899 | 0.7809 | −1.3875 | 0.0033 | −1.0976 | 0.0159 | 2 |
| 204822_at | −0.5371 | 0.5065 | −1.3949 | 0.0034 | −0.8579 | 0.0504 | 2 |
| 219990_at | −0.5251 | 0.5239 | −1.4035 | 0.0035 | −0.8784 | 0.0475 | 2 |
| 204444_at | −0.5381 | 0.5673 | −1.4631 | 0.0048 | −0.9250 | 0.0567 | 2 |
| 209681_at | −0.1770 | 0.8817 | −1.4845 | 0.0023 | −1.3074 | 0.0070 | 2 |
| 217028_at | −0.3032 | 0.7002 | −1.4904 | 0.0008 | −1.1872 | 0.0045 | 2 |
| 203373_at | −0.4812 | 0.5225 | −1.5266 | 0.0012 | −1.0454 | 0.0148 | 2 |
| 219479_at | −0.5931 | 0.3889 | −1.5301 | 0.0012 | −0.9370 | 0.0234 | 2 |
| 219596_at | −0.2341 | 0.7852 | −1.6024 | 0.0006 | −1.3683 | 0.0021 | 2 |
| 206268_at | −0.5816 | 0.6684 | −1.7595 | 0.0078 | −1.1779 | 0.0625 | 2 |
| 205771_s_at | 0.9001 | 0.4579 | −1.8483 | 0.0084 | −2.7485 | 0.0018 | 2 |

TABLE 4

| | | | | DNA | | | |
|---|---|---|---|---|---|---|---|
| | NCBI Gene | | | SEQ | | Protein | |
| | (LocusLink) | Gene | Probe | ID | DNA | SEQ | Protein |
| Entry | Title | Symbol | Set ID | NO: | Accession | ID NO: | Accession |
| 1 | !! 602155016F1 | | 213350_at | 188 | BF680255.1 | | |
| | NIH_MGC_83 Homo | | | | | | |
| | sapiens cDNA clone | | | | | | |
| | IMAGE: 4296050 5′, | | | | | | |
| | mRNA sequence | | | | | | |
| 2 | serpin | SERPINE2 | 212190_at | 189 | NM_006216.2 | 190 | NP_006207.1 |
| | peptidase | | | | | | |
| | inhibitor, | | | | | | |
| | clade E | | | | | | |
| | (nexin, | | | | | | |
| | plasminogen | | | | | | |
| | activator | | | | | | |
| | inhibitor | | | | | | |
| | type 1), | | | | | | |
| | member 2 | | | | | | |
| 3 | carcinoembryonic | CEACAM1 | 206576_s_at | 191; | NM_001024912.1; | 192; | NP_001020083.1; |
| | antigen- | | | 193 | NM_001712.3 | 194 | NP_001703.2 |
| | related cell | | | | | | |
| | adhesion | | | | | | |
| | molecule 1 | | | | | | |
| | (biliary | | | | | | |
| | glycoprotein) | | | | | | |
| 4 | !! 601306794F1 | | 212044_s_at | 195 | BE737027.1 | | |
| | NIH_MGC_39 Homo | | | | | | |
| | sapiens cDNA clone | | | | | | |
| | IMAGE: 3641231 5′, | | | | | | |
| | mRNA sequence \|\| | | | | | | |
| | ENST00000314138 | | | | | | |
| | 1476 30/2003 | | | | | | |
| 5 | matrix | MMP7 | 204259_at | 196 | NM_002423.3 | 197 | NP_002414.1 |
| | metallopeptidase 7 | | | | | | |
| | (matrilysin, | | | | | | |
| | uterine) | | | | | | |
| 6 | carcinoembryonic | CEACAM1 | 211889_x_at | 193; | NM_001712.3; | 194; | NP_001703.2; |
| | antigen- | | | 198 | D12502.1 | 199 | BAA02063.1 |
| | related cell | | | | | | |
| | adhesion | | | | | | |
| | molecule 1 | | | | | | |
| | (biliary | | | | | | |
| | glycoprotein) | | | | | | |
| 7 | !! clone | | 216246_at | 200 | AF113008.1 | | |
| | FLB0708 | | | | | | |
| | mRNA | | | | | | |
| | sequence | | | | | | |
| 8 | aldo-keto | AKR1C3 | 209160_at | 201 | NM_003739.4 | 202 | NP_003730.4 |
| | reductase | | | | | | |
| | family 1, | | | | | | |
| | member C3 | | | | | | |
| | (3-alpha | | | | | | |
| | hydroxysteroid | | | | | | |
| | dehydrogenase, | | | | | | |
| | type II) | | | | | | |
| 9 | !! NHTBCae03g02r1 | | 217685_at | 203 | AA853175.1 | | |
| | Normal Human | | | | | | |
| | Trabecular Bone Cells | | | | | | |
| | Homo sapiens cDNA | | | | | | |
| | clone | | | | | | |
| | NHTBCae03g02, | | | | | | |
| | mRNA sequence | | | | | | |
| 10 | carcinoembryonic | CEACAM1 | 209498_at | 191; | NM_001024912.1; | 192; | NP_001020083.1; |
| | antigen- | | | 193 | NM_001712.3 | 194 | NP_001703.2 |
| | related cell | | | | | | |
| | adhesion | | | | | | |
| | molecule 1 | | | | | | |
| | (biliary | | | | | | |
| | glycoprotein) | | | | | | |
| 11 | UDP | UGT1A1; | 208596_s_at | 204; | NM_000463.2; | 205; | NP_000454.1; |
| | glucuronosyl | UGT1A3; | | 206; | NM_001072.2; | 207; | NP_001063.2; |
| | transferase 1 | UGT1A4; | | 208; | NM_007120.2; | 209; | NP_009051.1; |
| | family, | UGT1A5; | | 210; | NM_019075.2; | 211; | NP_061948.1; |
| | polypeptide | UGT1A6; | | 212; | NM_019076.4; | 213; | NP_061949.3; |
| | A1; UDP | UGT1A7; | | 214; | NM_019077.2; | 215; | NP_061950.2; |
| | glucuronosyl | UGT1A8; | | 216; | NM_019078.1; | 217; | NP_061951.1; |
| | transferase 1 | UGT1A9; | | 218; | NM_019093.2; | 219; | NP_061966.1; |
| | family, | UGT1A10 | | 220; | NM_021027.2; | 221; | NP_066307.1; |
| | polypeptide | | | 222 | NM_205862.1 | 223 | NP_995584.1 |
| | A3; UDP | | | | | | |
| | glucuronosyl | | | | | | |
| | transferase 1 | | | | | | |
| | family, | | | | | | |
| | polypeptide | | | | | | |
| | A4; UDP | | | | | | |
| | glucuronosyl | | | | | | |
| | transferase 1 | | | | | | |
| | family, | | | | | | |
| | polypeptide | | | | | | |
| | A5; UDP | | | | | | |
| | glucuronosyl | | | | | | |
| | transferase 1 | | | | | | |
| | family, | | | | | | |
| | polypeptide | | | | | | |
| | A6; UDP | | | | | | |
| | glucuronosyl | | | | | | |
| | transferase 1 | | | | | | |
| | family, | | | | | | |
| | polypeptide | | | | | | |
| | A7; UDP | | | | | | |
| | glucuronosyl | | | | | | |
| | transferase 1 | | | | | | |
| | family, | | | | | | |
| | polypeptide | | | | | | |
| | A8; UDP | | | | | | |
| | glucuronosyl | | | | | | |
| | transferase 1 | | | | | | |
| | family, | | | | | | |
| | polypeptide | | | | | | |
| | A9; UDP | | | | | | |
| | glucuronosyl | | | | | | |
| | transferase 1 | | | | | | |
| | family, | | | | | | |
| | polypeptide | | | | | | |
| | A10 | | | | | | |
| 12 | CD55 | CD55 | 201926_s_at | 224 | NM_000574.2 | 225 | NP_000565.1 |
| | molecule, | | | | | | |
| | decay | | | | | | |
| | accelerating | | | | | | |
| | factor for | | | | | | |
| | complement | | | | | | |
| | (Cromer | | | | | | |
| | blood group) | | | | | | |
| 13 | plasticity- | MBP | 220798_x_at | 226 | NM_024888.1 | 227 | NP_079164.1 |
| | related gene 2 | (*) | | | | | |
| 14 | Extracellular | ECM1 | 209365_s_at | 228; | NM_004425.2; | 229; | NP_004416.1; |
| | matrix | | | 230 | NM_022664.1 | 231 | NP_073155.1 |
| | protein 1 | | | | | | |
| 15 | integrin, | ITGA6 | 215177_s_at | 232 | NM_000210.2 | 233 | NP_000201.2 |
| | alpha 6 | | | | | | |
| 16 | Coiled-coil | CCDC40 | 220593_s_at | 234; | XM_371082.3; | 235; | XP_371082.1; |
| | domain | | | 236; | XM_930250.2; | 237; | XP_935343.1; |
| | containing | | | 238; | XM_934521.2; | 239; | XP_939614.1; |
| | 40 | | | 240; | XM_942690.2; | 241; | XP_947783.1; |
| | | | | 242 | XM_946231.2 | 243 | XP_951324.1 |
| 17 | Chemokine | CXCL14 | 218002_s_at | 244 | NM_004887.3 | 245 | NP_004878.2 |
| | (C—X—C | | | | | | |
| | motif) ligand | | | | | | |
| | 14 | | | | | | |
| 18 | Ras-related | RRAD | 204803_s_at | 246 | NM_004165.1 | 247 | NP_004156.1 |
| | associated | | | | | | |
| | with | | | | | | |
| | diabetes | | | | | | |
| 19 | PR domain | PRDM12 | 220894_x_at | 248 | NM_021619.2 | 249 | NP_067632.2 |
| | containing | | | | | | |
| | 12 | | | | | | |
| 20 | ATP-binding | ABCB6 | 203191_at | 250; | AF308472.1; | 251; | AAG33617.1; |
| | cassette, | | | 252 | NM_005689.1 | 253 | NP_005680.1 |
| | sub-family B | | | | | | |
| | (MDR/TAP), | | | | | | |
| | member 6 | | | | | | |
| 21 | Death- | DAPK1 | 203139_at | 254 | NM_004938.2 | 255 | NP_004929.2 |
| | associated | | | | | | |
| | protein | | | | | | |
| | kinase 1 | | | | | | |
| 22 | Meiosis- | MNS1 | 219703_at | 256 | NM_018365.1 | 257 | NP_060835.1 |
| | specific | | | | | | |
| | nuclear | | | | | | |
| | structural 1 | | | | | | |
| 23 | coiled-coil | CCDC41 | 219644_at | 258; | NM_001042399.1; | 259; | NP_001035858.1; |
| | domain | | | 260 | NM_016122.2 | 261 | NP_057206.2 |
| | containing | | | | | | |
| | 41 | | | | | | |
| 24 | NIMA | NEK2 | 204641_at | 262 | NM_002497.2 | 263 | NP_002488.1 |
| | (Never in | | | | | | |
| | mitosis gene | | | | | | |
| | a)-related | | | | | | |
| | kinase 2 | | | | | | |
| 25 | chromosome | C14orf130 | 218108_at | 264 | NM_018108.2 | 265 | NP_060578.2 |
| | 14 open | | | | | | |
| | reading | | | | | | |
| | frame 130 | | | | | | |
| 26 | GINS | GINS1 | 206102_at | 266 | NM_021067.3 | 267 | NP_066545.3 |
| | complex | | | | | | |
| | subunit 1 | | | | | | |
| | (Psfl | | | | | | |
| | homolog) | | | | | | |
| 27 | BUB1 | BUB1 | 209642_at | 268 | NM_004336.2 | 269 | NP_004327.1 |
| | budding | | | | | | |
| | uninhibited | | | | | | |
| | by | | | | | | |
| | benzimidazoles 1 | | | | | | |
| | homolog | | | | | | |
| | (yeast) | | | | | | |
| 28 | RMI1, RecQ | RMI1 | 218979_at | 270 | NM_024945.1 | 271 | NP_079221.1 |
| | mediated | | | | | | |
| | genome | | | | | | |
| | instability 1, | | | | | | |
| | homolog (S. cerevisiae) | | | | | | |
| 29 | hypoxia- | HIG2 | 218507_at | 272 | NM_013332.1 | 273 | NP_037464.1 |
| | inducible | (*) | | | | | |
| | protein 2 | | | | | | |
| 30 | discs, large | DLG7 | 203764_at | 274 | NM_014750.3 | 275 | NP_055565.2 |
| | homolog 7 | | | | | | |
| | (Drosophila) | | | | | | |
| 31 | Fukuyama | FCMD | 205283_at | 276 | NM_006731.2 | 277 | NP_006722.2 |
| | type | | | | | | |
| | congenital | | | | | | |
| | muscular | | | | | | |
| | dystrophy | | | | | | |
| | (fukutin) | | | | | | |
| 32 | TTK protein | TTK | 204822_at | 278 | NM_003318.3 | 279 | NP_003309.2 |
| | kinase | | | | | | |
| 33 | E2F | E2F8 | 219990_at | 280 | NM_024680.2 | 281 | NP_078956.2 |
| | transcription | | | | | | |
| | factor 8 | | | | | | |
| 34 | Kinesin | KIF11 | 204444_at | 282 | NM_004523.2 | 283 | NP_004514.2 |
| | family | | | | | | |
| | member 11 | | | | | | |
| 35 | Solute | SLC19A2 | 209681_at | 284 | NM_006996.1 | 285 | NP_008927.1 |
| | carrier | | | | | | |
| | family 19 | | | | | | |
| | (Thiamine | | | | | | |
| | transporter), | | | | | | |
| | member 2 | | | | | | |
| 36 | Chemokine | CXCR4 | 217028_at | 286; | NM_001008540.1; | 287; | NP_001008540.1; |
| | (C—X—C | | | 288 | NM_003467.2 | 289 | NP_003458.1 |
| | motif) | | | | | | |
| | receptor 4 | | | | | | |
| 37 | Suppressor | SOCS2 | 203373_at | 290 | NM_003877.3 | 291 | NP_003868.1 |
| | of cytokine | | | | | | |
| | signaling 2 | | | | | | |
| 38 | KDEL (Lys- | KDELC1 | 219479_at | 292 | NM_024089.1 | 293 | NP_076994.1 |
| | Asp-Glu- | | | | | | |
| | Leu) | | | | | | |
| | containing 1 | | | | | | |
| 39 | THAP | THAP10 | 219596_at | 294 | NM_020147.2 | 295 | NP_064532.1 |
| | domain | | | | | | |
| | containing | | | | | | |
| | 10 | | | | | | |
| 40 | Left-right | LEFTY1 | 206268_at | 296 | NM_020997.2 | 297 | NP_066277.1 |
| | determination | | | | | | |
| | factor 1 | | | | | | |
| 41 | A kinase | AKAP7 | 205771_s_at | 298; | NM_004842.2; | 299; | NP_004833.1; |
| | (PRKA) | | | 300; | NM_016377.2; | 301; | NP_057461.1; |
| | anchor | | | 302 | NM_138633.1 | 303 | NP_619539.1 |
| | protein 7 | | | | | | |

### CEACAM1 (Carcinoembryonic Antigen-Related Cell Adhesion Molecule 1)

3 probe sets for CEACAM1 (206576_s_at, 211889_x_at, 209498_at) all showed significant increase in expression by compound 1 treatment at 17 days, but not by bevacizumab treatment (Table 3A). CEACAM family of proteins has been implicated in various intercellular-adhesion and intracellular signaling-mediated effects that govern the growth and differentiation of normal and cancerous cells. CEA-family members have crucial roles in vascular neogenesis, insulin metabolism, tumor development, and apoptosis, and as receptors for pathogenic bacteria and viruses.

CEACAM1 is expressed in microvessels of proliferating tissues, in tissues after wounding, and in solid human tumors. CEACAM1 stimulates proliferation, chemotaxis, and tube formation in microvascular human endothelial cells (C. Wagener et al., Exp Cell Res., November 25; 261(1):19-24 (2000)). CEACAM1-overexpressing microvascular endothelial cells showed prolonged survival and increased tube formation when they were stimulated with vascular endothelial growth factor (VEGF), whereas CEACAM1 silencing via small interfering RNA blocks these effects. In CEACAM1-overexpressing microvascular endothelial cells there is an up-regulation of angiogenic factors such as VEGF, VEGF receptor 2, angiopoietin-1, angiopoietin-2, tie-2, angiogenin, and interleukin-8 but a down-regulation of collagen XVIII/endostatin and Tie-1. These results suggest that constitutive expression of CEACAM1 in microvascular endothelial cells switches them to an angiogenic phenotype, whereas CEACAM1 silencing apparently abrogates the VEGF-induced morphogenetic effects during capillary formation (N. Kilic et al., J. Biol. Chem., January 21; 280(3):2361-9 (2005)).

One noteworthy point is that CEACAM1 down-regulation in prostate or bladder cancer cell lines is inversely correlated with its up-regulation in adjacent blood vessels. Supernatant of CEACAM1-overexpressing tumor cells suppressed but that of CEACAM1-silenced increased the VEGF-induced endothelial tubes (D. Tilki et al., Oncogene, March 27 (2006); L. Oliveira-Ferrer et al., Cancer Res., December 15; 64(24):8932-8 (2004)). This indicates that epithelial down-regulation and endothelial up-regulation of CEACAM1 act synergistically and lead to activation of angiogenesis and thus promotes tumor vascularization and invasion.

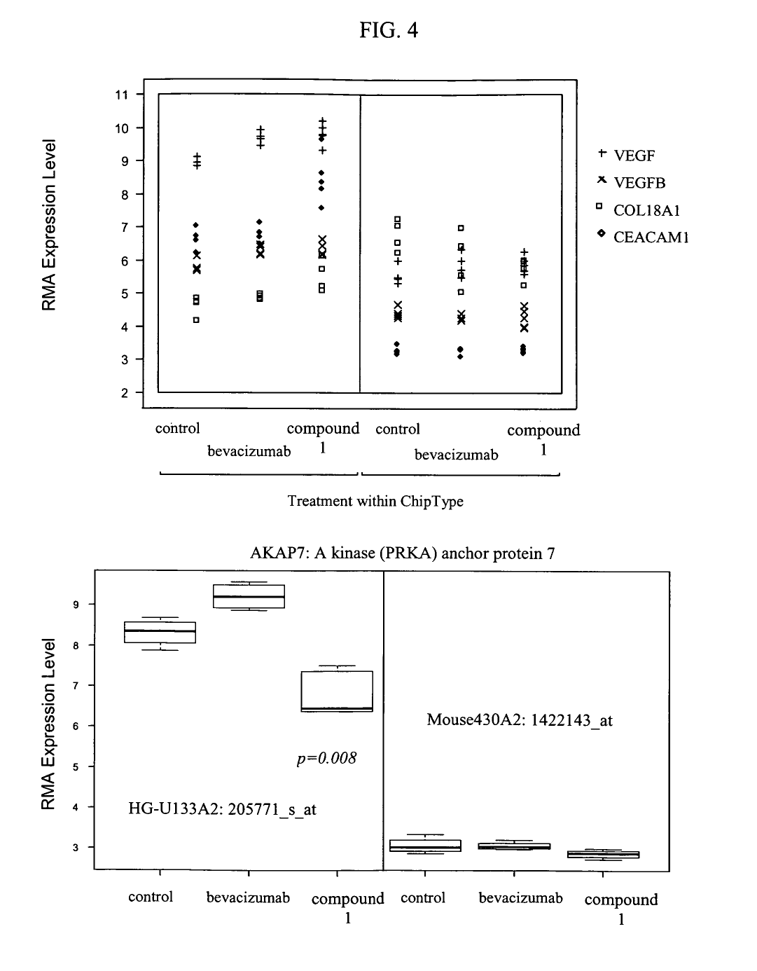

In our experiment, VEGF and VEGFB were both up-regulated by compound 1 at 17 days, along with up-regulation of CEACAM1. Expression of Collagen 18, the maternal substance of the angiogenesis inhibitor endostatin, was also up-regulated under the same condition (FIG. 4). Such up-regulation only happened in xenografts tumor tissues, but not in mouse stroma cells. The expression levels of these four genes were also significantly correlated with each other. One possible explanation of such expressional changes is that compound 1 induces CEACAM1 expression in tumor cells, which in turn suppresses VEGF-induced endothelial tube formation. Such angiogenesis suppression by CEACAM1 may cause compensatory up-regulation of VEGFs. On the other hand, expression of CEACAM1 in mouse epithelial cells did not change, possibly due to the anti-angiogenesis effect of compound 1 which inhibits tumor progression and invasion.

Endostatin, a 20-kd proteolytic fragment of collagen XVIII, is a major constituent of blood vessels throughout the body. The up-regulation of Endostatin, which has been shown to have antiangiogenic and antitumor activity before, also blocks endothelial migration and reduces VEGF-induced endothelial tube formation. This is consistent with the anti-angiogenesis activities of compound 1.

### AKAP7 (A Kinase (PRKA) Anchor Protein)

This gene encodes a member of the A-kinase anchoring protein (AKAP) family, a group of functionally related proteins that bind to a regulatory subunit (RII) of cAMP-dependent protein kinase A (PKA) and target the enzyme to specific subcellular compartments. AKAPs have a common RII-binding domain, but contain different targeting motifs responsible for directing PKA to distinct intracellular locations. In cell culture, interference of human AKAP7 mRNA in Hela cells by siRNA decreases survival of Hela cells (J. MacKeigan et al., J. Nat Cell Biol., June; 7(6):591-600 (2005)). In our experiment, AKAP7 was inhibited 3.5 fold (FDR q<0.008) upon compound 1 treatment at 17 days (FIG. 4). On the other hand, bevacizumab increased the expression levels of this protein. This indicated that compound 1 may have an advantage in promoting cell apoptosis compared to bevacizumab.

### Example 2

### Production of Antibodies Against the Biomarkers:

Antibodies against the biomarkers can be prepared by a variety of methods. For example, cells expressing an biomarker polypeptide can be administered to an animal to induce the production of sera containing polyclonal antibodies directed to the expressed polypeptides. In one aspect, the biomarker protein is prepared and isolated or otherwise purified to render it substantially free of natural contaminants, using techniques commonly practiced in the art. Such a preparation is then introduced into an animal in order to produce polyclonal antisera of greater specific activity for the expressed and isolated polypeptide.

In one aspect, the antibodies of the invention are monoclonal antibodies (or protein binding fragments thereof). Cells expressing the biomarker polypeptide can be cultured in any suitable tissue culture medium, however, it is preferable to culture cells in Earle's modified Eagle's medium supplemented to contain 10% fetal bovine serum (inactivated at about 56° C.), and supplemented to contain about 10 g/l nonessential amino acids, about 1,00 U/ml penicillin, and about 100 μg/ml streptomycin.

The splenocytes of immunized (and boosted) mice can be extracted and fused with a suitable myeloma cell line. Any suitable myeloma cell line can be employed in accordance with the invention, however, it is preferable to employ the parent myeloma cell line (SP2/0), available from the ATCC. After fusion, the resulting hybridoma cells are selectively maintained in HAT medium, and then cloned by limiting dilution as described by Wands et al. (1981, Gastroenterology, 80:225-232). The hybridoma cells obtained through such a selection are then assayed to identify those cell clones that secrete antibodies capable of binding to the polypeptide immunogen, or a portion thereof.

Alternatively, additional antibodies capable of binding to the biomarker polypeptide can be produced in a two-step procedure using anti-idiotypic antibodies. Such a method makes use of the fact that antibodies are themselves antigens and, therefore, it is possible to obtain an antibody that binds to a second antibody. In accordance with this method, protein specific antibodies can be used to immunize an animal, preferably a mouse. The splenocytes of such an immunized animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce the formation of further protein-specific antibodies.

### Example 3

### Immunofluorescence Assays:

The following immunofluorescence protocol may be used, for example, to verify VEGFR-2 biomarker protein expression on cells or, for example, to check for the presence of one or more antibodies that bind VEGFR-2 biomarkers expressed on the surface of cells. Briefly, Lab-Tek II chamber slides are coated overnight at 4° C. with 10 micrograms/milliliter (μg/ml) of bovine collagen Type II in DPBS containing calcium and magnesium (DPBS++). The slides are then washed twice with cold DPBS++ and seeded with 8000 CHO-CCR5 or CHO pC4 transfected cells in a total volume of 125 μl and incubated at 37° C. in the presence of 95% oxygen/5% carbon dioxide.

The culture medium is gently removed by aspiration and the adherent cells are washed twice with DPBS++ at ambient temperature. The slides are blocked with DPBS++ containing 0.2% BSA (blocker) at 0-4° C. for one hour. The blocking solution is gently removed by aspiration, and 125 μl of antibody containing solution (an antibody containing solution may be, for example, a hybridoma culture supernatant which is usually used undiluted, or serum/plasma which is usually diluted, e.g., a dilution of about 1/100 dilution). The slides are incubated for 1 hour at 0-4° C. Antibody solutions are then gently removed by aspiration and the cells are washed five times with 400 μl of ice cold blocking solution. Next, 125 μl of 1 μg/ml rhodamine labeled secondary antibody (e.g., anti-human IgG) in blocker solution is added to the cells. Again, cells are incubated for 1 hour at 0-4° C.

The secondary antibody solution is then gently removed by aspiration and the cells are washed three times with 400 μl of ice cold blocking solution, and five times with cold DPBS++. The cells are then fixed with 125 μl of 3.7% formaldehyde in DPBS++ for 15 minutes at ambient temperature. Thereafter, the cells are washed five times with 400 μl of DPBS++ at ambient temperature. Finally, the cells are mounted in 50% aqueous glycerol and viewed in a fluorescence microscope using rhodamine filters.

What is claimed is:

1. A method for identifying a mammal that will respond therapeutically to a method of treating cancer comprising administering an VEGFR-2 modulator, wherein the method comprises:
(a) measuring in the mammal the level of at least one biomarker selected from the biomarkers of Tables 2-4;
(b) exposing the mammal to the VEGFR-2 modulator;
(c) following the exposing of step (b), measuring in the mammal the level of the at least one biomarker,
wherein a difference in the level of the at least one biomarker measured in step (c) compared to the level of the at least one biomarker measured in step (a) indicates that the mammal will respond therapeutically to said method of treating cancer.

2. The method of claim 1 wherein the method is an in vitro method, and wherein the at least one biomarker is measured in at least one mammalian biological sample from the mammal.

3. A method for identifying a mammal that will respond therapeutically to a method of treating cancer comprising administering an VEGFR-2 modulator, wherein the method comprises:
(a) exposing the mammal to the VEGFR-2 modulator;
(b) following the exposing of step (a), measuring in the mammal the level of the at least one biomarker selected from the biomarkers of Tables 2-4,
wherein a difference in the level of the at least one biomarker measured in step (b), compared to the level of the biomarker in a mammal that has not been exposed to said VEGFR-2 modulator, indicates that the mammal will respond therapeutically to said method of treating cancer.
